(19) United States
(12) Patent Application Publication
(10) Pub. No.: US 2024/0125796 A1
(43) Pub. Date: Apr. 18, 2024

(54) DETECTION OF BIOMARKERS

(71) Applicant: REFEYN LTD, Oxford (GB)

(72) Inventors: Ana Sofia De Jesus FERREIRA, Oxford (GB); Matthias Karl Franz Langhorst, Oxford (GB)

(21) Appl. No.: 18/187,250

(22) PCT Filed: Sep. 21, 2021
(86) PCT No.: PCT/GB2021/052451

§ 371 Date: Mar. 21, 2023

(30) Foreign Application Priority Data
Sep. 21, 2020 (GB) 2014880.5

(51) Int. Cl. G01N 33/68; G01N 21/47; G01N 33/574

(52) U.S. Cl. CPC G01N 33/6854; G01N 21/4738; G01N 33/57415; G01N 2333/71

(57) ABSTRACT

A method and kit is described for the detection of a biomarker in a sample, by light scattering microscopy. The invention is particularly useful for the detection of low abundance biomarker in complex samples, and is suitable for use in a point of care setting. In an embodiment, the method of the invention describes detection of a biomarker in a sample using mass photometry.

The present invention relates to the use of light scattering, in particular interferometric scattering microscopy or mass photometry, for the detection or quantification of a biomarker in a sample. In particular, the present invention relates to a method of diagnosis of a disease or condition associated with a biomarker in a sample. Also provided are kits for performing a method of the invention. Since the sample may be complex, the biomarker may be present as a minor component, in the presence of other components. The biomarker therefore may be present in low concentrations.

### BACKGROUND

The detection or quantification of biomarkers has become widely utilised as a tool for diagnosis of a disease or condition, or determining susceptibility of an individual to a certain disease condition. Biomarker detection is an invaluable tool in the growth of personalised medicine, enabling targeted treatment regimens to those patients testing positive for the presence or absence of an indicative biomarker, or panel of biomarkers. For example, cancer, autoimmune or inflammatory diseases can be diagnosed by detecting abnormal presence of proteins or other biomarkers in samples taken from body fluids such as blood, CSF, and urine. Such biomarkers can be the presence or absence of specific proteins, proteins isoforms, post-translation modifications, abnormal quantities of protein(s) or changes in the relative ratios of protein to protein/metabolites or protein to other biomolecules (e.g. sugar, polysaccharides, nucleic acids).

Biomarkers include a broad spectrum of cellular, biochemical and/or molecular alterations that can be evaluated, monitored or measured and that have been linked to a health condition, a pathogenic process or a response to treatment or medical intervention. Biomarkers are commonly used as diagnostic of several diseases in relative advanced stages, but their potential in the detection of early stages where treatment or preventive actions can be more effective is still very limited, mostly because early disease biomarkers are not abundant and exist in very low concentrations. Most common methods to detect low abundance biomarkers include targeted or quantitative mass spectrometry, and aptamer based proteomics.

Alongside the growth in identification of suitable biomarkers has been the development of methods, systems and hardware for detecting or quantifying a biomarker. Most commonly used methods are enzyme-linked immunosorbent assays (ELISA) and polymerase chain reaction (PCR)-based tests, which offer high sensitivity, but either have to be optimized for rapid non-quantitative testing or take between 30 minutes to several hours to complete and are thus too complex and/or expensive for use in a point-of-care setting.

Other approaches such as electrochemical immunoassays, surface-enhanced Raman spectroscopy, flow cytometry or other fluorescence-based technologies have been developed to address the shortcomings of ELISA, but so far none have reached the ideal combination of a simple, affordable instrument with high specificity and sensitivity.

In recent years, interferometric scattering microscopy (iSCAT), most notably mass photometry has been developed as a powerful analytical technology for single-molecule detection, offering a simple and cost-efficient alternative to assays such as ELISA. iSCAT provides information about the relative distribution of particles of different masses in solution, without the requirement to add a label. iSCAT has been described previously for detection of purified single proteins (Cole et al (ACS Photonics, 2017, 4(2), pp 211-216 and WO2018/011591), and for the detection of lipoproteins and determination of concentrations of a molecule in solution (WO2019/110977). Mass photometry in particular has been described in Young et al, Science, Apr. 2018: Vol. 360, Issue 6387, 423-427) and Li et al, Nucleic Acids Research, August 2020, https://doi.org/10.1093/nar/gkaa632.

Interferometric scattering mass spectrometry (iSCAMS), herein denoted mass photometry (MP), is a means for detecting and measuring the mass of single objects and the complexes they form in solution. MP detects single molecules by their light scattering as they bind non-specifically or specifically to a surface. Each binding event leads to a change in refractive index at the surface/solution interface, which effectively alters the local light scattering and can be detected with high accuracy by taking advantage of optimized interference between scattered and reflected light. The magnitude of the signal change can be converted into a molecular mass, for polypeptides with ˜2% mass accuracy and up to 20 kDa mass resolution by calibration with molecules of known mass. The scattering signal is thus directly proportional to the molecule's mass; making it possible to weigh single molecules with light. However, such a technique cannot be simply applied to complex solutions where one molecule may be present in low abundance. Thus, despite the fact that the molecule of low abundance may bind non-specifically to the surface and permit a detection of mass, there may be too many confounding components present to make an accurate determination of mass and therefore identity of the molecule. This makes identification of said molecule complicated and a determination of concentration difficult. The application of this technique, therefore, to low abundance biomarkers in samples has therefore been limited.

Mass photometry is unique in its capability for accurate mass measurement of single molecules in solution, in their native state and without the need for labels.

Light scattering has long been used in biochemical analyses, such as the use of a measuring the light-scattering species in solution by means of the light intensity at an angle away from the incident light passing through the sample, known as “nephelometry”. Other, more refined methods exist that rely on the principles of light scattering, such as dynamic light scattering (DLS) (measures the size and size distribution of particles), Electrophoretic Light Scattering (ELS) (measures electrophoretic mobility and zeta potential), and Low-Angle Laser-Light Scattering (LALLS) (allowing measurement of the molecular weight distribution of polymers in a sample). However, all differ from the light scattering methods and apparatus used in the present invention, since the technique used enables a direct mass measurement of a single molecule (object) at a surface. Notably, techniques that rely upon principles of light scattering such as surface plasmon resonance depend entirely upon the binding of multiple particles to induce a bulk change in refractive index in the assay, which cannot provide the level of detail required to determine the presence, concentration and individual mass of a biomarker in a complex solution.

There is still a lack of sensitivity and specificity in detection of biomarkers used for disease progression, disease regression or rapid clinical screening for potential drug candidates. By way of example, one of the most common biomarkers is C-reactive protein (CRP), but this biomarker is highly non-specific and can indicate a variety of different conditions and diseases. Improving the analysis of more specific biomarkers routinely accessible would be a turning point in routine diagnostics. Furthermore, biomarkers for neurological diseases and cancer are often present in body fluids at early stages of the disease, but their concentration might be too low to be accurately detected by the routinely used techniques available at point-of-care. To be suitable for a point of care setting, there is a further need for reduction in the cost, time and skills required to obtain assay results whilst maintaining accuracy.

There therefore remains a need for a specific and sensitive method which enables quantitative detection of a very low abundancy biomarker in dirty environments including debris or large aggregates, such as biological samples, and which is further suitable for a point of care setting.

### BRIEF SUMMARY OF THE DISCLOSURE

The present inventors have surprisingly identified a methodology which enables the specific detection of biomarkers, particularly those present at low abundancy in a sample, using light scattering as defined herein. The present inventors have shown that it is possible to detect low abundance biomarkers in a complex sample, without the need for lengthy purification procedures. The method developed by the present inventors is particularly suitable for detection of a biomarker in a biological sample, for example for use in the diagnosis of a disease or condition. It is further particularly suitable for monitoring industrial and agricultural samples for biomarkers, such as monitoring food production systems for pathogens or sewerage for outbreak of viral disease, for example.

Given the ability of interferometric light scattering microscopy and indeed mass photometry to determine the mass and concentration of a molecule in solution simply by it binding non-specifically to a surface, the inventors have developed a counterintuitive method which instead relies on specific binding of a biomarker to a surface by utilising a capture agent. However, instead of measuring this binding (as would be routine for such determinations), the inventors determined that this did not produce sufficiently accurate or quantitative results, particularly if other components were present in the sample and thus a low abundance species would only represent very few binding events among a large background of non-specific binding events. Therefore, they realised that by effectively determining the mass of an object at the surface before and after part or all of it is released (as a particle) would permit an indirect measurement of the mass of the particle—independent of non-specific binding of much more abundant species in the sample. Given the accurate detection methods, the present invention may be used if the biomarker is released from the capture agent, or if indeed the capture agent or a part thereof is released with the capture agent. This enables flexibility in the choice of capture agent and indeed release agent.

Since the biomarker is likely present at low abundancy and also in the presence of other components of the sample, routine detection of concentration via binding is not currently possible. This is because the other components may bind non-specifically to the surface and provide confounding data. The elegant solution to this is to specifically capture the biomarker, remove confounding components via washing and then detect the release of the biomarker by monitoring the surface for a change or difference in light scattering. That difference may be assigned a mass and the identity of the biomarker can then be assigned.

Accordingly, in a first aspect, there is provided a method of detecting a biomarker in a sample, the method comprising contacting a surface comprising a capture agent for the biomarker immobilised thereon with the sample, releasing any captured biomarker from the surface, and detecting release of the biomarker by light scattering.

Light scattering is detected in an interferometric light scattering microscope or a mass photometer.

Suitably, there is provided a method for detecting a biomarker in a sample, wherein the method comprises:
I) providing a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface;
IV) releasing a particle bound to the surface, wherein the particle is selected from:
   i) the biomarker released from the capture agent to which it is bound;
   ii) a complex comprising the biomarker bound to the capture agent; and/or
   iii) unbound capture agent;
V) detecting the particle(s) released from the first detection area of the surface by light scattering.

The particle(s) are detected by detecting a change in the light scattering at the surface. The release event may therefore require at least two measurement events; one measurement of the surface before the release event and one after.

The measurement before and after the release event enables a negative mass event to be detected. The mass of the particle released can be determined using the methods of the invention, by virtue of recording the change at the surface. This permits a direct determination of if the specific biomarker is present.

Suitably, there is provided a method for detecting a biomarker in a sample, wherein the method comprises:
I) providing a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface and taking a measurement of the surface using light scattering;
IV) releasing a particle bound to the surface, wherein the particle is selected from:
   i) the biomarker released from the capture agent to which it is bound;
   ii) a complex comprising the biomarker bound to the capture agent; and/or
   iii) unbound capture agent;
VI) detecting the particle(s) released from the first detection area of the surface by determining the change in light scattering.

The measurement of the surface may be a measurement of object(s) at the surface. The surface is preferably a single surface. The surface may be any appropriate conformation, since the nature of the surface is irrelevant to the measurements taking place. In the Examples, the surface is a flat glass coverslip.

The light scattering method may be interferometric scattering microscopy. The light scattering method may be mass photometry.

The first detection area may be the observable surface, or the area contacted by the release agent.

Effectively, the present invention radically changes the way interferometric light scattering (iSCAT) is used, by an entire reversal of the way the measurement is achieved. iSCAT and similar techniques are generally applied to samples that are substantially pure or with minimal “impurities” (such as other molecules not of interest). In order to detect “rare” species in a sample, various techniques such blocking the surface to prevent other species binding have been tried. Even with a passivated surface, unspecific binding may occur. The novel approach described here permits the detection of a rare species in a complex sample because the non-specific binding event is not monitored, these species can be washed away and then the method entirely concentrates on the unbinding event which is specific to the rare species.

The release of a particle, including a single biomarker, biomarker/capture agent complex or unbound capture agent, from the surface may be referred to as a release event. Suitably, the step of releasing a particle from the surface may be performed at a rate which allows for detection of a single release event by light scattering. Light scattering microscopy is capable of determining the mass of particles released from the surface into solution, the identity of which can be determined by knowledge of their mass. Effectively, the loss of mass is detected at the surface. Using light scattering microscopy to detect a biomarker released from the surface into solution enables the specific detection of low abundancy biomarkers in a highly sensitive manner. The method has the advantage of avoiding the need for labelling of a biomarker or biomarker/capture agent complex. Furthermore, it allows the capture of mass heterogeneity of a biomarker population, information which is not accessible by any other technique.

Preferably, in light scattering microscopy, optionally mass photometry, the surface may be described as interrogated or examined almost constantly, such as via a near continuous repetitive rate. This is generally performed using controlled illumination of the surface described herein. The light scattering device detects single molecules via light scattering at the surface. Thus, the present invention may monitor the surface and detect object(s) at the surface (which may be the capture agent alone, the capture agent bound to the biomarker or even additional components from the sample). A release event may be caused by the addition of a release agent. Each release event leads to a change in the local light scattering (for example of the object) which can be detected with high accuracy taking advantage of optimised interference between scattered and reflected light. The magnitude of any change in signal can be converted to molecular mass.

Thus, the mass of the particle released is detected. This detection is indirect.

Preferably, the detection of a change at a location or of an object at a location is possible because a measurement is taken before and after the release event. In simplest terms, this means that two measurements are taken in order to detect the release event. The release event may therefore by detected as a negative mass event: the departure of the mass is detected. Thus, the measurement permits detection of an object at the surface and then the release of a particle from that object. The identity of the particle released can be determined by virtue of calculating the mass difference before and after the release event. FIG. 5 is an example of the “negative masses” detected. The method may therefore involve the monitoring of a specific object or objects for a change. The method may therefore involve the monitoring of a specific location or locations for a change. The change may be a change in light scattering indicating a release of the particle.

The rate at which the release event is performed may be controlled, such that it is possibly to monitor the negative mass events as they occur.

The magnitude of any change in signal is converted to a molecular mass for that change.

Any modifications to the biomarker may be detected using the method of the invention, for example glycosylation, attachment of nucleic acids, or ubiquitination. Detection of “post translational” or other modifications may be due to a specific molecule weight being associated with the modified biomarker.

A method of the first aspect method may be used to determine the concentration of a biomarker in a sample. The method may therefore comprise measuring the amount of biomarker released from the capture agent and/or the amount of the biomarker/capture agent complex released from the surface on which it is immobilised. The method may be used to determine concentration by comparison with a calibration curve. To construct a calibration plot, a series of calibration solutions are made containing known concentrations of reference standard. A calibration curve may be constructed using a control which includes a known number of release events. A calibration curve may be used to obtain a measure of quantity or concentration of a biomarker, biomarker/capture agent complex or unbound capture agent.

An internal standard may also be used. As is routine to analytical methods, an internal standard is a chemical substance added in a constant amount to the surface for either/both calibration and biomarker detection assays. The internal standard can then be used for calibration by plotting the ratio of the biomarker signal to the internal standard signal as a function of the biomarker concentration.

The contrast detected in a release event may then be correlated with a second mass calibration curve to identify their mass. To construct a mass calibration plot, a series of calibration solutions are made containing particles of known mass. Where mass photometry is used, the particles can be identified by mass and a quantification of the single biomarker, biomarker/capture agent complex or unbound capture agent can be obtained using a calibration curve. Measuring the amount of unbound capture agent released from the surface may be used to quantify the proportion of capture agent occupied by biomarker present in the sample, and provide a quantitative result.

The step of releasing the biomarker from the capture agent and/or the biomarker/capture agent complex or unbound capture agent from the surface may comprise disrupting the binding between the biomarker and the capture agent, and/or disrupting the binding between the capture agent and the surface upon which the capture agent is immobilised. The disruption is suitably sufficient to fully dissociate the biomarker from the capture agent and/or the capture agent from the surface. Any suitable means or methods for mediating release of a biomarker and/or capture agent may be used, as described herein. Suitable methods include changing the chemical environment of the surface, enzymatic digestion of the capture agent and/or photolysis or hydrolysis. Suitable means of releasing a biomarker includes the introduction of a competitive ligand with a higher binding affinity for the capture agent that promotes the release event.

The method may further comprise comparing the mass of the biomarker/capture agent complex released to the expected mass of the biomarker. Such a method may be useful in detection of a biomarker in a heterogenous biomarker population.

A method of the first aspect may further comprise repeating steps IV) and V) for a second or further detection area of the surface. The repetition of steps IV) and V) for a second or further detection area may be in a same or different observation event to steps IV) and V) performed on the first or any previous detection area of the surface. Suitably, the release of the biomarker and/or biomarker/capture agent complex may be restricted to the detection area of interest in any particular observation event.

Prior to step I), a method of the invention may further comprise capturing a biomarker present in a sample on a surface. Such capture may be specific, thereby serving enrich the biomarker on the surface. A method of the invention may therefore comprise:
i) providing a surface
ii) providing a sample to be analysed for presence and/or amount of a biomarker;
iii) immobilising on the surface a capture agent which specifically binds to the biomarker to be detected;
iv) incubating the surface of iii) with the sample for a suitable time period and under suitable conditions to allow binding of the biomarker present in the sample to the capture agent immobilised on the surface.

Optionally, following step (iv) the surface may be washed to remove unbound entities and remove entities which have non-specifically bound to the surface or the capture agent. The washing conditions are selected such that the interaction between the capture agent and the biomarker is unaffected. Suitable conditions include the inclusion of a mild detergent or low concentration of salt solutions. Alternatively, a drying step may be included, prior to the addition of a suitable solution for the detection step. This drying step may include using a flow of gas such as nitrogen over the surface. Such a washing and optional drying step may be labelled step (v).

Such steps (i) to (v) may be carried out outside of any light scattering apparatus.

Such steps (i) to (v) may be carried out prior to the illumination of the surface.

Once the surface has been contacted with the sample and appropriate preparation steps such as (i) to (v) are carried out, the surface may be (vi) contacted with a buffer to ensure that the surface is in solution.

Any of steps (i) to (vi) may be conducted prior to any detection steps, in any appropriate order.

A method of the invention may further comprise performing a base-line measurement of light scattering of the surface. Such a step may be performed after any one or more of steps i) to iv) above. A baseline measurement may serve as a control measurement. A baseline measurement may provide information on any object(s) immobilised on the surface, by virtue of their light scattering. A baseline measurement may be used to determine light scattering from the surface to allow for any corrections. Furthermore, a baseline measurement may provide a measurement of random release events, providing a baseline above which the release events are expected to be specific and worthwhile of detection.

A method of the invention may involve multiple measurement steps, which may commence once the surface is present in a light scattering apparatus, i.e. all the necessary preparation steps described above have been performed. Thus, measurement may commence at any point after step (iii) described above. These multiple measurements may be discrete (for example at time intervals) or the measurements may effectively be continuous (measurements taken at a constant rate in order to generate a film—see later). Multiple measurements may permit the determination of the mass of object(s) at the surface and therefore detect the release of any particle from said object. Thus, this permits detection of the release event. The detection of the release event may therefore be described as detecting the mass of an object before and after a release event and calculating the difference in mass of that object. Thus, a specific object or object(s) is monitored for a change in mass. A change in the magnitude of a signal for an object is therefore detected.

A method of the invention may further comprise generating a calibration curve using a control molecule with a known number of release events.

A method of the first aspect of the invention may be useful for detecting or quantitating a biomarker in a sample.

A method of the first aspect may be useful for detecting modification of a biological molecule, for example post-translational modification of a protein or methylation status of a nucleic acid. The modified biological molecule may be the biomarker.

A method of the first aspect may be useful for detecting interactions between two or more biological molecules in a sample. The complex between the biological molecules may be the biomarker.

A method of the first aspect may be useful for comparing the amounts of two or more biomarkers in a sample.

Also provided is a method of detecting contamination in a sample, for example a cell culture or a food preparation sample, comprising the method of the first aspect wherein the presence of the biomarker is indicative of contamination of the sample.

Also provided is a method of quantitating the expression level of a product, wherein the method comprises a method of the first aspect and wherein the biomarker is indicative of presence or absence of the expression product.

Also provided is a method of detecting or quantitating interaction between biological molecules in a sample, comprising the method of the first aspect wherein the presence or amount of the biomarker is indicative of presence or absence or amount of interaction. Such a method may be useful in detecting the presence or amount of a compound which affects the presence or activity of the biomarker.

In a second aspect, the present invention provides a method of diagnosing a disease or condition associated with presence, absence or amount of the biomarker in a subject, wherein the method comprises contacting a surface comprising a capture agent for the biomarker immobilised thereon with the sample, releasing any captured biomarker from the surface, and detecting release of the biomarker by light scattering. The amount may be a relative amount in comparison to another biomarker.

Suitably, a method of diagnosing a disease or condition associated with presence or amount of the biomarker in a subject comprises:
I) providing in solution a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface;
IV) releasing a particle bound to the surface, wherein the particle is selected from:
   i. the biomarker released from the capture agent to which it is bound;
   ii. a complex comprising the biomarker bound to the capture agent; and/or
   iii. unbound capture agent;
V) detecting the particle(s) released from the first detection area of the surface by light scattering,

wherein the presence or absence of i) or ii) is indicative of the presence, severity, or likelihood of developing the disease or condition in the subject.

The particle(s) are detected by detecting a change in the light scattering at the surface. The release event may therefore require at least two measurement events; one measurement of the surface before the release event and one after.

Suitably, a method of diagnosing a disease or condition associated with presence or amount of the biomarker in a subject comprises:
I) providing in solution a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface and taking a measurement of the surface using light scattering;
IV) releasing a particle bound to the surface, wherein the particle is selected from:
i. the biomarker released from the capture agent to which it is bound;
   ii. a complex comprising the biomarker bound to the capture agent; and/or
   iii. unbound capture agent;
VI) detecting the particle(s) released from the first detection area of the surface by determining the change in light scattering,

wherein the presence or absence of i) or ii) is indicative of the presence, severity, or likelihood of developing the disease or condition in the subject.

The measurement of the surface may be a measurement of object(s) at the surface. The surface is preferably a single surface. The surface may be any appropriate conformation, since the nature of the surface is irrelevant to the measurements taking place. In the Examples, the surface is a flat glass coverslip.

Suitably, a method of determining the presence/absence of a contamination associated with presence or amount of the biomarker in a sample comprises:
I) providing in solution a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface;
IV) releasing a particle bound to the surface, wherein the particle is selected from:
i. the biomarker released from the capture agent to which it is bound;
   ii. a complex comprising the biomarker bound to the capture agent; and/or
   iii. unbound capture agent;
V) detecting the particle(s) released from the first detection area of the surface by light scattering,

wherein the presence or absence of i) or ii) is indicative of the presence, severity, or nature of the contamination.

The particle(s) are detected by detecting a change in the light scattering at the surface. The release event may therefore require at least two measurement events; one measurement of the surface before the release event and one after.

Suitably, a method of determining the presence/absence of a contamination associated with presence or amount of the biomarker in a sample comprises:
I) providing in solution a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface and taking a measurement of the surface using light scattering;
IV) releasing a particle bound to the surface, wherein the particle is selected from:
   i. the biomarker released from the capture agent to which it is bound;
   ii. a complex comprising the biomarker bound to the capture agent; and/or
   iii. unbound capture agent;
V) detecting the particle(s) released from the first detection area of the surface by determining the change in light scattering,

wherein the presence or absence of i) or ii) is indicative of the presence, severity, or nature of the contamination.

The measurement of the surface may be a measurement of object(s) at the surface. The surface is preferably a single surface. The surface may be any appropriate conformation, since the nature of the surface is irrelevant to the measurements taking place. In the Examples, the surface is a flat glass coverslip.

The light scattering method in either or any of these methods may be interferometric scattering microscopy or mass photometry.

An optional step of washing the surface after contacting the surface with a sample may be included. Suitable washing conditions are described herein.

An optional step of drying the surface may be included, prior to contacting the surface with a solution prior to the detection step.

A method of the invention may further comprise performing a base-line measurement of light scattering of the surface. Such a step may be performed after step (II) above. A baseline measurement may serve as a control measurement. A baseline measurement may provide information on any object(s) immobilised on the surface, by virtue of their light scattering. Furthermore, a baseline measurement may provide a measurement of random release events, providing a baseline above which the release events are expected to be specific and worthwhile of detection.

As used herein the object may be any object at the surface, such as capture agent without or without associated biomarker, and even other components from the sample.

A method of the invention may include any suitable internal standard or control. An internal control may be used to determine relative concentrations. A method of the invention may include any suitable internal calibrant.

The method may be used to determine the concentration of a biomarker in a sample. The method may therefore comprise measuring the amount of biomarker released from the capture agent and/or the amount of the biomarker/capture agent complex released from the surface on which it is immobilised. The amount of biomarker may be used as an indication of progression or severity of a disease or condition.

The release of the biomarker from the capture agent and/or the biomarker/capture agent complex or unbound capture agent from the surface may be as described herein, and in relation to the first aspect.

A method of the second aspect may include one or more additional steps, as described herein, in particular in relation to the first aspect.

A method of the second aspect may be used for determining the risk that the subject will develop said disease or condition.

A method of the second aspect may be suitable for selecting a subject to whom a substance or composition is to be administered, or to whom a treatment or dosage regimen is to be prescribed, wherein said substance or composition or regimen is suitable for treating or preventing a disease or condition associated with the presence or amount of a biomarker in a sample from the subject. A subject may be selected for administration of the substance or composition, or treatment or dosage regimen if the presence or absence or amount of biomarker in the sample is indicative of the presence or likelihood of developing the disease or condition.

The second aspect also provides a method of treating or preventing a disease or condition in a subject diagnosed with a disease or condition or likelihood of developing said disease or condition in the subject according to the second aspect, wherein the method comprises administering a substance or composition to the subject, or carrying out a regimen thereon, which is effective to treat or prevent said disease or condition in the subject.

Also provided is a substance or composition for use in a method of treating or preventing a disease or condition in a subject diagnosed with a disease or condition or likelihood of developing said disease or condition in the subject according to the second aspect.

Also provided is the use of a substance or composition in the manufacture of a medicament for treatment or prevention of a disease or condition associated with presence, absence or amount of a biomarker, wherein the subject has been diagnosed with a disease or condition or likelihood of developing said disease or condition in a method according to the second aspect.

In a third aspect, there is provided a kit, wherein the kit comprises means suitable for carrying out a method of the first or second aspect of the invention. A kit may comprise one or more items selected from: a suitable surface, a capture agent, a buffer, a calibration chart, instructions for use in accordance with a method of the invention, a sample collection device, an agent to mediate release of the biomarker or capture agent as described herein, and one or more standard biomarker samples for calibration.

### BRIEF DESCRIPTION OF THE DRAWINGS

Embodiments of the invention are further described hereinafter with reference to the accompanying drawings, in which:

FIG. 1 is a diagrammatic illustration of a method of the present invention;

FIG. 2 shows the results of a method of the invention performed as described in Example 1. The first graph shows the control measurement (number of particles released identified by mass), and the second graph shows the amount of Her2 released from the coated coverslip after addition of hydrochloric acid to decrease the pH of the TBS buffer, mass of particles released;

FIG. 3 shows the possible arrangement of an apparatus for use in iSCAT and suitable for use in the present invention;

FIG. 4 is a diagrammatic illustration of a method of the present invention, this is exemplified in Example 2; and

FIG. 5 shows the results of the experimentation in Example 2. This is a graph that shows the mass of the released particles from the surface (as the mass is released, a negative figure is shown).

### DETAILED DESCRIPTION

The present inventors have identified a method to use single particle light scattering, preferably single particle interferometric scattering microscopy or mass photometry to detect a biomarker in a sample. In particular, the present inventors have surprisingly found that it is possible to utilise light scattering microscopy to detect low abundance biomarkers in a complex sample. Typically in the art, detection of a biomarker, even at low abundance, is performed by detecting a binding event, either to a support or to a binding partner such as an antibody or receptor protein. The present invention is based upon a novel approach taken by the present inventors, which provides, for the first time, a highly specific and sensitive assay suitable for the detection of low abundance biomarkers in a complex sample. Rather than detecting a binding event, the present inventors have found that by releasing a biomarker from a surface to which it is bound, and detecting the release using light scattering microscopy, low abundance biomarkers can be detected in a specific and sensitive manner. The present invention is therefore based on the novel approach of detecting unbinding, or release, of a particle from a surface.

Such a method can be used for the detection of any biomarker in a sample, and has numerous applications, for example in methods of medical diagnosis, industrial processes, and quality control. Suitably, a method of the invention is as described in the first and second aspects of the invention. Also provided is a kit, as described in the third aspect of the invention.

The following terms have, for the purposes of this application, the meanings as provided below. Unless otherwise defined, all technical and scientific terms used herein have the same meanings as commonly understood by a person skilled in the art.

Sample

A sample may be any biological, industrial, or environmental sample. A sample may be a simple or complex solution. Biological samples include samples taken or obtained from a human or animal body or individual, for example blood, serum, plasma, urine, saliva, lymph, sweat, amniotic fluid, cerebrospinal fluid, breast milk, tears, secretions, synovial fluid, semen, bile, or mucus, a lung fluid sample; and a stool sample. A bodily fluid may be capillary, venous or arterial blood or plasma or serum therefrom. Suitably, a blood sample, such as a sample of blood derived from a finger prick, may be used in a method of the invention. In certain embodiments a saliva sample, such as a Buccal swab of saliva, may be used. Bodily fluids are examples of complex solutions where numerous solutes are present, including electrolytes, sugar and urea. A biological sample may be a clinical sample. A biological sample may be a tissue sample obtained from a human or animal body or individual. A biological sample may be a cell culture sample, for example bacterial or viral culture, including phage culture. A biological sample may be from an in vitro source. Samples may be prepared prior to analysis using standard techniques, such as tissue homogenisation and cell lysis.

If the sample is an environmental sample, it may be taken from any source, such as water (for example wells, streams, rivers, lakes, rainwater, seawater and the like), or waste products such as sewerage, agricultural effluent and the like.

Industrial samples may be taken food and drinks (for example beverages), agricultural samples or liquid samples from factories and manufacturing processes. Industrial samples may also include samples from bioreactors and laboratories to check for biological contamination of cell preparations and the like.

Different types of samples may be processed simultaneously, sequentially or separately by a method or device of the invention. In certain embodiments only one type of sample may be used at once. In alternative embodiments, two or more samples may be integrated or combined, and used in the same method.

Samples might require routine pre-treatment, such as cell lysis, removal of cell debris or any other pre-treatment that is common practice in other diagnostic techniques. Therefore, a method of the invention may comprise preparing the sample for use. A sample may also require dilution, for example in a suitable buffer. Typically, a suitable buffer has a physiological pH and salt concentration.

A method of the invention may comprise a step of obtaining a sample from a subject, or from an industrial or environmental source, using any suitable method. Suitable methods are known in the art and may include collection of saliva, sputum or urine, a swab test, finger prick test, arterial or venous blood sampling by needle, lumbar puncture, lung aspiration, biopsy, amniocentesis, paracentesis, or throacocentasis.

A method of the invention may comprise a step of obtaining a sample from a cell culture or from an industrial process, or from the environment. A method of the invention may comprise the step of obtaining a sample of sewerage water.

The sample volume required for detection of a biomarker by light scattering is minimal, and may be as little as a microlitre, depending on the sample type and means of collection.

The sample may be collected or provided in any suitable sample chamber. A suitable chamber may be a specimen container, vial, bottle, ampoule, test tube, Eppendorf tube, microcentrifuge tube, capillary tube or bag. A method of the invention may comprise storage of a sample. A method of the invention may comprise transfer of a sample or part thereof to a surface, upon which a method of the invention is performed.

Subject

The term “subject,” may be used interchangeably with the terms “individual” and “patient” and refer to an animal subject, suitably a vertebrate subject, and even more suitably a mammalian subject. Suitable vertebrate animals include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards, etc.), and fish. A preferred subject is a primate (e.g., a human, ape, monkey, chimpanzee).

A subject may be an individual suffering from one or more symptoms of a disease or condition. A subject may be an individual who has been categorised as requiring testing for predisposition to a disease or condition, or for a likelihood of developing a particular disease or condition.

Biomarker

As used herein, the term “biomarker” refers to any biological feature from a sample to be detected or quantitated. A biomarker may be virtually any biological compound, such as a protein and a fragment thereof, a peptide, a polypeptide, a proteoglycan, a glycoprotein, a lipoprotein, a carbohydrate, a lipid, a nucleic acid, an organic or inorganic chemical, a natural polymer, and a small molecule, that may be present in the sample to be analysed. A biomarker may be a polynucleotide, such as a deoxyribose nucleic acid or a ribonucleic acid, such as a pathogenic genome or fragment thereof. A biomarker may be a combination, or conjugate of two or more of the above. A suitable biomarker for detection according to the present invention is capable of being bound or immobilised on a surface, for example via a capture agent.

A biomarker can be useful or potentially useful for measuring the initiation, progression, severity, pathology, aggressiveness, grade, activity, disability, mortality, morbidity, disease sub-classification or other underlying feature of one or more biological processes, pathogenic processes, diseases, conditions or responses to a therapeutic intervention. Diagnosis as referred to herein includes making such a measurement.

According to their possible applications, biomarkers can be classified as different groups that include among others: diagnostic biomarkers, monitoring biomarkers, pharmacodynamic/response biomarkers, predictive, prognostic, safety and susceptibility/risk biomarkers, and quality control markers (Califf, R. M. Biomarker definitions and their applications. Exp. Biol. Med. 243, 213-221 (2018)).

As used herein, the term “nucleic acid” refers to a polymer or oligomer of nucleotide or nucleoside monomers consisting of naturally occurring bases, sugars and inter-sugar linkages. The term “nucleic acid” also includes polymers or oligomers comprising non-naturally occurring monomers, or portions thereof, which function similarly. A nucleic acid can be DNA, RNA or chimeric, i.e., comprising both deoxy- and ribo-nucleotides. A biomarker may be a modified form of a nucleic acid, for example a methylated form thereof.

A carbohydrate may include monosaccharides, disaccharides, oligosaccharides, and polysaccharides, and modified forms thereof, such as presence of specific acetyl-, acyl- or aryl-groups.

A protein as used herein may include peptides, polypeptides, post-translationally modified proteins, e.g. by ubiquitination, lipidation, phosphorylation, alkylation or glycosylation. The methods of the invention may be used to determine if the biomarker is post-translationally modified.

Biomarkers also include fats including lipids, fatty acids, monoglycerides, diglycerides, triglycerides, phospholipids, glycerolipids, glycerophospholipids, sphingolipids, and saccharolipids.

Also included are small molecules including, without limitation, drugs and metabolites thereof, hormones, neurotransmitters, metabolites, and vitamins.

Other biological molecules which may be a biomarker include glycopeptides, glycoproteins, glycolipids, waxes, sterols, fat-soluble vitamins, and liopoproteins.

Also included are clusters of molecules, assemblies, aggregations, protein/protein interactions, protein/small molecule interactions, protein-nucleic acid interactions, protein-sugar interactions; and/or oligomeric assemblies.

Derivatives or metabolites of any of the above entities may also be considered to be biomarkers.

Examples of biomarkers include, without limitation:

Cancer—AFP (liver cancer), BCR-ABL (chronic myeloid leukaemia), BRCA1/BRCA2 (breast/ovarian cancer), BRAF V600E (melanoma/colorectal cancer), CA-125 (ovarian cancer), CA19.9 (pancreatic cancer), CEA (colorectal cancer), EGFR (non-small-cell lung cancer), HER-2 (breast cancer), KIT (gastrointestinal cancer), PSA (prostate specific antigen), S100 (melanoma).

Cardiovascular disease—BNP and NT-proBNP for diagnosis of heart failure and/or heart failure exacerbation; troponin for diagnosis and risk stratification of patients with suspected acute coronary syndrome; CRP to assess the risk of cardiovascular disease, heart attack, and stroke; circulating levels of MPO to predict risks of coronary heart disease (Huang, et al Dis. Markers 2017, 2-4 (2017); Truemper, et al Biomarkers Cardiovasc. Dis. 27, 1-20 (2015).

Autoimmune disease—extensive biomarkers are known and available in the field, and reviewed for example in Norouzinia, et al Gastroenterol. Hepatol. from Bed to Bench 10, 155-167 (2017); Jin, F. et al. Front. Immunol. 9, 1-9 (2018); Shi, G., et al J. Immunol. Res. 2017, 1-2 (2017); Prince, H. E. et al Biomarkers 10 Suppl 1, 44-49 (2005)). Bacterial infections—CRP, white blood cell (WBC) count, polymorphonuclear (PMN) leucocyte count and PCT.

Viral infections (e.g. early stages of Epstein-Barr virus or acute HIV infection) can be distinguished from bacterial infections by measuring CD64 and CD169.

Liver injuries—serum levels of alanine aminotransferase, aspartate aminotransferase, alkaline phosphatase, and gamma-glutamyl transferase.

Suitably, the biomarker and/or the biomarker/binding agent complex is not labelled for the purpose of detection according to the present invention. Therefore, it is not necessary to include any form of label for either the capture agent or the biomarker. The method of the invention may therefore be described as label-free.

A biomarker may be present at low or very low concentrations in a sample. A sample may be a complex mixture of both high and low concentration biomarkers, wherein the latter may vary in their concentration level from microgram per millilitre level through concentrations in the picogram per millilitre range; down to femtogram per millilitre level..

Herein, a biomarker may also include a set of two or more pre-defined biomarkers. A set may include 2, 3, 4, 5, 10, or 20 or more biomarkers. This set of pre-defined biomarkers may be related to the same disease/condition for example, and as such be described as a panel, such that a complete diagnosis or prognosis is possible using a single test. In any multiplexed set, it is feasible to use a different release mechanism for the different capture agents.

Surface

A surface for use in the present invention may be any suitable surface which may be functionalised for enrichment of the biomarker in the sample, and is compatible with the chosen detection method. A suitable surface may be capable of having bound thereto or immobilised thereon a capture agent.

A surface may comprise one or more detection areas. A detection area may be any suitable size and shape. Suitably, a detection area is the area which can be analysed by a light scattering microscope at the same time, or in a single observation event. The size and shape of a detection area may be guided by the size and shape of the area illuminated during light scattering, and therefore by the type of microscope used. A surface may comprise more than one detection area. Where more than one detection areas are provided on a surface, they may be adjacent, or may overlap. Where more than one detection areas are provided, they may be spaced apart from one another. A surface may comprise 1, 2, 3, 4, 5, 10, 20, 50, 70, 100, 500 or 1000 or more detection areas.

Preferably, the surface is planar or substantially planar. The surface may be curved or include some curvature, for example a concave depression or convex structure on a substantially planar surface. The surface is preferably not the surface of a nanoparticle, since small spherical objects would cause light scattering in their own right and prevent an accurate determination of mass of the release event. A nanoparticle or ultrafine particle is usually defined as a particle of matter that is between 1 and 100 nanometres in diameter, optionally between 1 and 60 nanometres in diameter, optionally less than 50 nanometres in diameter. Thus, small spherical particles with a diameter of 60 μm or below, optionally 50 μm or below, optionally 40 μm or below, are optionally not used as a surface according to the present invention. A substantially planar surface may be preferred.

A detection area may be pre-defined. Alternatively, a detection area may be defined during performance of the method. For example, a detection area may be defined by the flow of a release agent over a surface, such that a detection area is defined fully or partially by the area contacted by the release agent. The detection area may be defined by the concentration of release agent. The detection area may be defined by the illumination area of the photolysis illumination. If an alternative release mechanism is chosen, the interplay between release and observation area will determine the detection area.

A surface is suitably solid (i.e. not a gel or a liquid). Suitably a detection area of a surface allows transmission of ultraviolet light (which may be defined herein as having wavelengths in the range from 10 nm to 380 nm); visible light (which may be defined herein as having wavelengths in the range from 380 nm to 740 nm); and/or infrared light (which may be defined herein as having wavelengths in the range from 740 nm to 300 μm).

Suitably, a detection area allows transmission of light in the visible light spectrum. Suitably, a detection area is substantially transparent.

A detection area of a surface may be smooth or may be textured. A textured surface, for example a mesh or knitted or woven fabric may increase the binding capacity of the detection area. The remainder of the surface excluding a detection area may be smooth or may be textured, and may be the same or different to the detection area. A smooth surface is preferred.

A surface or a detection area thereof may be of any suitable material, including for example but without limitation glass, diamond, plastic, polymeric material (e.g. cyclic olefin copolymer, polyvinyl, polyethylene (PE) including for example polyethylene terephthalate (PET) and high-density polyethylene (HDPE) and low-density polyethylene (LDPE), polyacrylate (acrylic), polystyrene (PS) including high impact polystyrene (HIPS), silicone, polyester (for example polylactic acid (PLA) or polylactic coglycolic acid (PGLA)), polyurethane, polypropylene (PP), polyamide (nylon), Acrylonitrile butadiene styrene (ABS), Polyethylene/Acrylonitrile Butadiene Styrene (PE/ABS), bakelite, rubber, latex, polycarbonate (PC), Polycarbonate/Acrylonitrile Butadiene Styrene (PC/ABS) and polyvinyl chloride including for example polyvinylidene chloride (PVDC), and sapphire.

Any two or more detection areas of a surface may be of the same or different in terms of one or more of material, texture, size, shape, and functionalisation.

A surface may have any suitable geometry. For example, a surface may comprise a flat plate, such as a coverslip, or may be a well, plate, channel, container, flow cell, flow chamber, microfluidic cell or chamber, or slide. A surface may be part of a larger geometry or device. A suitable surface may be a glass coverslip or a plastic coverslip, wherein the plastic is as described above in relation to the surface.

A surface preferably forms part of a sample holder. Said sample holder may be an element of a light scattering microscope. The sample holder may be a high surface-to-volume chamber.

A surface or indeed a detection area may be functionalised. A surface or detection area may be passivated, activated, coated, treated or derivatised. The surface may be a passivated surface. Passivation is the process of treating or coating a surface in order to enhance or reduce the chemical reactivity, thus increasing or decreasing the number of binding events. Those skilled in the art will be aware of suitable passivating agents, examples of which include BSA, PVPA, DDS and/or PEG. The use of lipid layers, such as monolayers and bilayers on the surface is contemplated. Alternatively, the surface is not passivated.

If the surface is coated, derivatised or modified, thin surface alterations may be desirable. Altered surface layers that are too thick can change the light scattering properties of the surface. Alteration of only the outermost few molecular layers (3-10 nm) may be desirable.

A surface or a detection area may be activated, coated, treated and/or derivatised to enable binding of a capture agent thereto. Functionalisation of a surface or detection area enables the selection of the biomarker from the sample, effectively enriching the biomarker on the surface. Functionalisation of a detection area may comprise i) modifying the surface to prevent any non-specific binding, ii) modifying the surface to be capable of binding a capture agent; and iii) binding a capture agent to the modified surface. Suitably, capture agent bound to the detection area will be functionally orientated for binding to a biomarker. Functionalisation may be chemical (e.g. covalent bonding) or physical (e.g. adsorption).

Any suitable surface coating may be applied to enable binding of a capture agent. Suitable methods and coatings will be known and available to persons skilled in the art, including for example and without limitation, functionalised PEG, silanes, amines, aminosilane, aldehyde modification with amine modification reagents such as APTES, epoxy modification using for example GOPTS, carboxylate modification using for example EDC, NHS, HOBt, TBTU, PAMAM; diazo using for example NaNO₂, and supramolecules for example calixarenes. Suitable methods are known and available in the art to immobilise a capture agent on a surface.

Where a surface or detection area is functionalised as described herein to enable binding of a capture agent, the functionalisation or binding of a capture agent does not significantly alter the ability to detect a biomarker as described herein. Suitably, a functionalisation process and/or binding of a capture agent does not substantially affect the light transmission through a treated detection area of a surface.

Functionalisation of a surface may be to permit various interactions with the capture agent, such as chemical interactions (e.g. covalent bonding) or physical interactions (e.g. adsorption). The capture agent may react chemically with the functional groups of the surface via free reactive groups such as amine or carboxyl groups, resulting in the formation of covalent bonds, immobilising the capture agent. Hydrophobic, hydrogen, van der Waals, ionic and coordinate bonding types, along with electrostatic interactions may also occur between the capture agent and the surface. These types of interaction may be particularly affected by the conditions, such that changes in temperature and pH may alter the interaction of the capture agent with the surface, enabling a release of the capture agent when the conditions are altered.

A surface may be treated in order to alter its hydrophobicity, for a hydrophobic coating can be applied to the surface. Such may permit immobilisation of the capture agent. Proteins and lipids, for example, may be described amphiphilic macromolecules since they possess both hydrophobic and hydrophilic groups attracted to nonpolar and polar groups, respectively. This may be used to enable the immobilisation of the capture agent.

The means for immobilisation of the capture agent to the surface can be selected based on the requirement for the release event. For example, if the capture agent can be permanently immobilised and the biomarker can be selectively released from the biomarker using a release agent, then the surface may be functionalised to permit permanent immobilisation, such that only the biomarker is released during the release event. Alternatively, if the capture agent has a high affinity for the capture agent, the method of immobilisation may be selected such that the capture agent (and any associated biomarker) may be released using the release agent, such as a change in pH.

The surface may be modified or functionalised to include binding partners for the capture agent in order to enable immobilisation. As exemplified, biotin can be immobilised to the surface by coating the surface with PEG and biotinylated PEG. Streptavidin as a capture agent may then be immobilised using its binding affinity for biotin. As streptavidin is a homo-tetramer, it is capable of still presenting binding sites for biotin in order to capture biotinylated biomarkers in the sample.

Capture Agent

Any suitable capture agent may be immobilised on a detection area of a surface, to capture biomarker of interest from a sample. A suitable capture agent will be capable of specifically binding the biomarker of interest. By specific binding is meant that the capture agent binds to the biomarker to be detected with greater affinity than it binds to other molecules under the same conditions. Specific binding is generally indicated by a dissociation constant of 1 μM or lower, e.g., 500 nM or lower, 400 nM or lower, 300 nM or lower, 250 nM or lower, 200 nM or lower, 150 nM or lower, 100 nM or lower, 50 nM or lower, 40 nM or lower, 30 nM or lower, 20 nM or lower, 10 nM or lower, or 1 nM or lower.

A capture agent may be an antibody or an antibody fragment that specifically recognizes the biomarker to be detected. A capture agent may be a protein, peptide or peptidomimetic that binds a protein or non-protein target (such as a protein that specifically binds to a ligand such as a small molecule biomarker, or a receptor that binds to a protein biomarker). A capture agent may be a nucleic acid such as an aptamer or ribozyme. A capture agent may be a nucleic acid that can hybridize to complementary sequences. A capture agent may be a polysaccharide, lipid, lipopolysaccharide, teichoic acid, or lipoteichoic acid that specifically binds to a biomarker. A capture agent may be an antigen or ligand which specifically binds to a biomarker which is an antibody or fragment thereof, or receptor. A capture agent may be naturally occurring or may be recombinant, or synthetic. A capture agent may not be entirely naturally occurring, and may comprise a portion thereof which is naturally occurring and a fragment or portion thereof which is recombinant or non-naturally occurring.

A detection area may comprise any suitable number of capture agents. The number of capture agents may depend upon the type and abundance of the biomarker to be detected. A detection area may comprise more than 1,000, or more than 10,000, or more than 100,000, or more than 500,000, or more than 1,000,000 capture agents specific for the biomarker of interest.

Binding between the biomarker and the capture agent may be non-covalent, such as one or more of hydrogen bonding, Van der Waals forces, electrostatic forces, hydrophobic forces, and the like. However, interaction or binding can also be covalent.

As used herein, the term “antibody” or “antibodies” refers to an intact immunoglobulin or to a monoclonal or polyclonal antigen-binding portion with the Fc (crystallizable fragment) region or FcRn binding fragment of the Fc region. The term “antibodies” also includes “antibody-like molecules”, such as portions of the antibodies, e.g., antigen-binding portions. Antigen-binding portions can be produced by recombinant DNA techniques or by enzymatic or chemical cleavage of intact antibodies. “Antigen-binding portions” include, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The antibody may be engineered.

A nucleic acid capture agent may be an agent capable of specifically binding the biomarker in a non-sequence dependent manner, such as an aptamer. Aptamers are generally short nucleic acid sequences that obtain a conformation capable of binding. Other nucleic acids that can bind include ribozymes and the like.

A nucleic acid capture agent may be a nucleic acid as defined herein, which has the ability to specifically bind to a biomarker to be detected. A nucleic acid capture agent may comprise a combination of non-specific sequence, and specific sequence. A nucleic acid capture agent may be naturally occurring or may be recombinant. A nucleic acid capture agent may be engineered to include one or more binding sequences specific for a biomarker to be detected. The specificity of the sequence may be such that the nucleic acid capture sequence binds to a nucleic acid biomarker or nucleic acid portion of a biomarker under stringent conditions.

The term “stringent conditions” refers to conditions under which a nucleic acid strand will hybridise preferentially to, or specifically bind to, its complementary binding partner and to a lesser extent to, or not at all to, other sequences. The term “stringent hybridization conditions” as used herein refers to conditions that are compatible to produce duplexes between complementary nucleic acid strands, e.g., between DNA probes and complementary targets in a sample or between a primer and a nucleic acid molecule to be amplified with a substantial lack of duplexes formed between non-complementary nucleic acid strands. Stringent conditions are known in the art, for example as defined in Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)).

A protein capture agent may be a protein, polypeptide or peptide, optionally conjugated to a nucleic acid, carbohydrate or lipid. A protein capture agent comprises a suitable binding site for a biomarker to be captured. A binding site of a protein capture agent may exhibit specific binding for a nucleic acid, protein, small molecule, lipid or carbohydrate biomarker. Examples of protein capture agents include receptors, transcription factors, toxins, anti-toxins, polysaccharides and polysaccharide derivatives. A suitable protein capture agent may bind to a biomarker to be detected with a dissociation constant which is in the nanomolar to picomolar range. It should be noted that the method of the invention may permit the use of capture agents that are not as highly specific as other techniques such as ELISAs, because the technique permits the identification of the bound species by weight. Thus, the use of capture agents with specificity of binding in the micromolar range is also possible, should the biomarker have an easily identifiable weight.

If the biomarker is itself capable of binding to entities, for example it is an antibody, enzyme or receptor, the capture agent for such may be the binding partner for the biomarker, such as an antigen, substrate or ligand.

A capture agent can be generated by any method known in the art. For example, antibodies can be found in an antiserum, prepared from a hybridoma tissue culture supernatant or ascites fluid, or can be derived from a recombinant expression system, as is well known in the art. Fragments, portions or subunits of e.g., an antibody, receptor or other species, can be generated by chemical, enzymatic or other means. The present invention also contemplates that capture agents may include recombinant, chimeric hybrid, humanised, primatised or other modified forms.

The capture agent may be a bioconjugate, comprising for example any suitable combination of biomolecules to serve as a capture agent. For example, protein-nucleic acid combinations, or antibody-protein conjugate, antibody-nucleic acid conjugates.

The capture agent may be a binding agent with a plurality of binding sites. Therefore, such a capture agent may be immobilised using its binding affinity. In the Examples, it is shown that streptavidin can be immobilised on a surface by virtue of the coating of a surface with biotinylated PEG. Streptavidin homo-tetramers have a high affinity for biotin (Kd ˜10⁻¹⁴ mol/L) and each subunit binds biotin with the same affinity. Thus, streptavidin immobilised on biotinylated PEG will still have binding sites available for biotinylated biomarkers in the Example. Since streptavidin has the highest affinity for free biotin, free biotin can be added as a release agent, releasing a particle from the surface, the departure of which may be detected.

An unbound capture agent as referred to herein is a capture agent which is not bound to the biomarker to be detected. Suitably, an unbound capture agent is not bound to any other biomolecule, either specifically or non-specifically. The capture agent may be unbound at the point it is measured at the surface prior to the release event (as an “object”). Thus, no biomarker has bound to the capture agent.

The release agent may bind to the capture agent (and any biomarker present) during release (such as biotin to streptavidin in Example 2). As it is the release event that is detected, rather than any signal from the released particle itself, the nature of the particle after release is not interrogated. Thus, the binding of any release agent is irrelevant.

A capture agent may comprise one or more cleavage site(s) for digestion by an enzyme or cleavage by photolysis or hydrolysis (change in pH). One or more cleavage sites may be provided in a biomarker binding site, such that upon cleavage, a bound biomarker is released. One or more cleavage sites may alternatively or additionally be provided such that upon cleavage the capture agent in a bound or unbound state is released from the surface. A cleavage site which releases the capture agent from the surface is preferably proximal to a surface binding site of the capture agent, such that substantially the whole capture agent is released from the surface. The cleavage sites may be naturally occurring in the capture agent, or may be artificially introduced, for example by recombinant technology. A cleavage site may be provided in a linker sequence introduced into the capture agent. Where the capture agent is a nucleic acid, a suitable sequence may be included for any DNA cleaving enzyme, such as a restriction enzyme site.

When the agent contains a cleavage site for photolysis, this may be any suitable photolysis site, which can be included in any suitable part of the capture agent, including the linker tethering the capture agent to the surface. The capture agent may comprise a photolabile protecting group (PPG, also known as: photoremovable, photosensitive, or photocleavable protecting group), for photosensitive cleavage. The PPG may be nitrobenzyl-, carbonyl- or benzyl-based. A simple photocleavable linker may be built into the capturing moiety.

Photolysis is not limited to visible light; this requires the use of any photon with sufficient energy. Thus, electromagnetic waves with the energy of visible light or higher, such as ultraviolet light, x-rays and gamma rays are usually involved in such reactions. The photolabile site can be selected such that the light used to cleave is suitable for use in conjunction with the illumination source for interferometric light scattering. Thus, UV light may be preferred, for example. Photocleavable sites are extensively used in chemical and biological sciences; because light can be delivered with very high spatiotemporal precision. Multiple capture agents using different photocleavable sites could be used on a single surface, using different wavelengths of light to release the various capture agents.

It will be understood that the use of a PPG does not prevent the capture agent from interacting with the biomarker. The PPG is simply present to permit the cleavage of the capture agent or a part thereof and release it from the surface.

Hydrolysis is any chemical reaction in which a molecule of water ruptures one or more chemical bonds. For example, the non-enzymatic cleavage rate of amide bonds located in peptides in aqueous solution is pH-dependent; an alkaline pH can promote breakage of the amide bonds. The capture agent may be designed such as to include a section which is more prone to hydrolysis. Suitable conditions would need to be selected to ensure that the biomarker was not also similarly broken down.

Examples of suitable cleavage sites include cleavage sites for proteins/antibodies, or the inclusion of restriction enzyme sites for nucleic acids. Enzymatic cleavage may also be performed using DNA or RNA enzymes (DNAzymes or RNAzymes). Such can be engineered to permit specific cleavage at a target site. Restriction sites are well known to those skilled in the art, and generally (for endonucleases) involve a specific sequence within a double stranded polynucleotide, which is about 6 to 8 nucleotides in length.

The capture agent may be released by disrupting the non-covalent interactions causing immobilisation. Thus, a change in pH or temperature may be sufficient to disrupt the immobilisation of the capture agent, and this can be the release event.

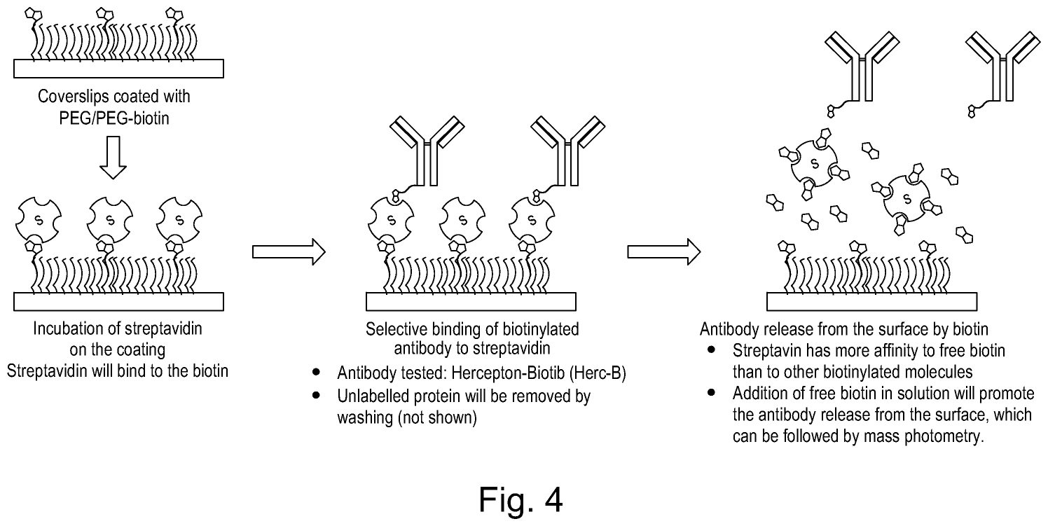

The capture agent may be released by providing a competitive binding partner therefore disrupting the binding to immobilised binding partners (see Example 5 and FIG. 4).

If the capture agent and means of immobilisation are selected such that the capture agent is released using a release agent, the methods of the invention can determine whether a biomarker was bound to the capture agent on the surface prior to the release event.

None, part or all of the capture agent may form the particle during the release event, depending upon the release mechanism used.

Contacting a Surface with the Sample

In an embodiment, a surface is contacted with the sample to allow binding of any biomarker present in the sample to the capture agent immobilised on the surface.

Any suitable means and methods may be used to contact the sample and the surface. Contacting the sample and the surface includes incubating exposing, mixing, or delivering, a sample to the surface. Contacting may require in certain embodiments agitating, vortexing, pipetting etc. Contact may be performed for a sufficient time period to allow binding of biomarker present in the sample to the capture agent immobilised on the surface. The contact time can be of any suitable length, depending on the binding affinities and/or concentrations of the capture agents or the biomarker, concentrations thereof, or incubation conditions (e.g., temperature). Contact time may be at least about 30 seconds, at least about 1 minute, at least about 5 minutes, at least about 10 minutes, at least about 15 minutes, at least about 30 minutes, at least about 1 hour, at least about 2 hours, at least about 3 hours, at least about 4 hours, at least about 6 hours, at least about 8 hours, at least about 10 hours, at least about 12 hours, at least about 24 hours, at least about 48 hours or longer. A person skilled in the art will be able to adjust the contact time and conditions accordingly.

Suitable hybridisation conditions will be known to the person skilled in the art, and will depend upon the nature of the biomarker and capture agent.

Washing

During preparation of a surface, non-specific binding may occur. In order to remove any molecules that bind non-specifically to the surface, the surface may be washed using a suitable method which does not substantially affect immobilisation of the capture agent or binding of the biomarker of interest thereto. Suitable washing buffers and methods will be known and available to a person skilled in the art, for example varying salt concentrations or mild detergents which will suitably ensure that only specific desired interactions will remain intact. Ideally, the washing step does not alter the conditions in which the capture agent is immobilised.

Buffer or Detection Solution

A surface which has been washed may then be contacted with a buffer or other suitable solution for the detection step, this is preferably a clean buffer. As used herein, the term “clean buffer” refers to a buffer which produces minimal background noise, such that it does not create results that interfere with the measurement, results or conclusion. Those skilled in the art will be aware that there many examples of buffers that can be suitable used in the detection step. Examples of clean buffers include Tris buffered saline, phosphate buffered saline, HEPES, MES, MOPS or MEM. A suitable solution is water. A surface, or a detection area thereof, may be fully or partially submerged. Suitably, at least a detection area being observed is fully submerged in the buffer or solution.

Release of the Biomarker and/or Complex from the Surface

The invention is based on the realisation that release of the biomarker from the surface at a controlled rate enables the identification of the biomarker, even at very low abundance in the sample. The release event may cause the biomarker to be released from the capture agent immobilised on the surface. It may additionally or alternatively cause the release of the capture agent (or part thereof) from the surface to which it is immobilised. The capture agent may be bound to the biomarker of interest, or may be unbound. The capture agent may remain bound to the biomarker after release or it may dissociate. The method of the invention is sensitive enough to measure both release of the biomarker as a particle and release of the capture agent and/or biomarker as a particle.

The release event is the change of binding at the surface/solution interface. Therefore, suitably a release event means complete detachment of the particle from the surface into the solution holding the surface. Once the particle is released from the surface, this release is detected. Such a release is detected by measuring the surface before and after the release event. An object, for example a capture agent (with or without associated biomarker), can be measured on the surface before and after any release of the particle. The difference in light scattering between the measurements detects the release event of the particle. Each release event leads to a change in the local light scattering (for example of the object) which can be detected with high accuracy. The magnitude of any change in signal can be converted to molecular mass. Thus, the mass of the particle is calculated from a before/after measurement from the surface. For the avoidance of doubt, the released particle itself is not detected by light scattering, since it has been released into the solution.

Thus, the present invention relates to the detection of the presence at the surface then absence of the particle from the surface. By calculating the difference in these measurements, a mass can be attributed to the particle. Effectively, therefore, an object or location in the detection area is monitored for a change in light scattering.

Thus, if the capture agent and the biomarker dissociate during or after the release event, this does not affect the detection of the release event, or calculation of mass released. If, for example, the biomarker is released first, followed by a release of the capture agent, this would be detected as two changes in light scattering at the surface, leading to a calculation that the biomarker was present at the surface prior to the release event, followed by release of the capture agent. Alternatively, if the particle released initially comprises the biomarker bound to the capture agent and these subsequently dissociate in solution, only the release of the particle is detected. What happens to the particle beyond the surface (in solution) is not measured or detected. The detection will be the same, since the event that is detected is the “unbinding” of the particle from the surface. What happens once the unbinding occurs is not detected, since the particle is no longer at the surface.

There are multiple particle types that may be released. These include the biomarker alone, the biomarker bound to a capture agent (or part thereof) or the unbound capture agent (or part thereof). The three particle types, namely any biomarker, unbound capture agent and/or bound capture agent (biomarker/capture agent complex) are released at a defined rate and under defined conditions, from the surface. Suitably, these particles are released into the solution in which the surface is in contact.

The rate of release is suitably selected to enable detection of individual release events by light scattering. Therefore, the present invention is capable of detecting individual particles released from the surface including any biomarker of interest, complex or unbound capture agents, and characterising the same. In this manner, a method of the present invention is able to assign a mass to an individual particle, based on the measurements obtained from the object before and after release of the particle from the surface. Release of the particle may result in the object being released from the surface (for example the unbound capture agent or capture agent/biomarker complex). Release of the particle may result in a portion of the object being released (for example a biomarker released from the capture agent, or a biomarker complexed to a portion of a capture agent with a portion left on the surface). Thus, determining the nature of the particle released is achieved by monitoring the object at the surface and determining what is present before and after the release event.

Suitably therefore, to maximise the sensitivity of a method of the invention, the release of bound particles from the surface is controlled such that it occurs at a rate which enables detection by light scattering to identify and characterise individual unbinding (release) events. This permits monitoring of individual objects at the surface for release of a particle.

Suitably, therefore, an individual particle is released at a time from the detection area. By this may be meant that the rate of release is such that no more than 100 release events occur every second, or within a single image frame capture. The rate of release may be up to 100, up to 90, up to 80, up to 70, up to 60, up to 50, or up to 40 release event per second. Therefore, whilst a single release event may be captured on one or more subsequent frames, a single frame suitably does not include more than 100 release events. This allows for individual detection of biomarker, capture agent or complex, and quantitation of individual release events. Where multiple release events occur substantially simultaneously, such that individual events cannot be distinguished in a frame (for example due to overlap of the illuminated spots), the release rate may be too high and an adjustment is needed to reduce the release rate. Essentially, whilst the image capture device of the light scattering microscope would be able to record the light scattering events associated with the release event, it would not be possible above a certain concentration to distinguish individual objects and assign a mass thereto. Therefore, put another way, a suitable release rate is one which provides an image capture frame in which any events do not overlap and therefore can be individually identified and characterised. The number of spots per frame, and therefore the release rate, will depend upon the size of the frame. An exposure time per frame may be 0.01 to 50 ms, suitably 0.5 to 20 ms.

A suitable time interval for detecting the release events for a detection area will depend upon various factors including the size of the detection area; the concentration of capture agents bound thereto; the abundance of any biomarker; the strength of the release agent and the rate of release; and the frame capture rate. The suitable time interval will therefore depend upon specific parameters for the detection event. However, it may be possible to impose a limit of a certain time or frames, such as 10,000 frames.

A release event may be triggered by any suitable method. Examples of suitable methods include:
i) Changing the chemical environment of the surface. A method of the invention may therefore comprise effecting a chemical change in the environment of the surface to trigger a release event. An example includes altering the pH of the solution containing the detection area. A change in pH is known to disrupt protein-protein interactions, such as antibody-antigen interactions. A reduction in pH may be preferred. For example, a change in chemical environment may include lowering the pH thereof, for example by addition of acid, such as hydrochloric acid. Similarly, altering the redox conditions in the environment of the detection area may also serve to trigger a release event. In relation to nucleic acids in particular, changing the ionic strength of the environment by the addition of an ion, for example, may be sufficient to trigger a release event. Any suitable means may be used to introduce a chemical change in the environment. Suitable means include exposure to light (e.g. by photolysis of caged protons which affect the pH); addition of a buffer or salt.
ii) Disrupting the binding between a biomarker and capture agent, and/or a capture agent and the surface by enzymatic cleavage. A suitable enzyme may be used to target a cleavage site in the binding site of the capture agent. The binding site may be the biomarker binding site or may be the surface binding site. Cleavage of a biomarker binding site may serve to release the biomarker from the capture agent. Cleavage of a surface binding site may serve to release the capture agent from the surface. The released capture agent may be unbound or may be bound to a biomarker. A suitable enzyme may be activated, for example by exposure to light. A suitable enzyme can be proteinaceous, or composed of nucleic acids or hybrids thereof.
iii) Digestion of the capture agent. Any suitable means may be applied to the surface to digest the capture agent, thereby releasing from the surface the biomarker. An enzyme may be used to digest the capture agent. For example if the capture agent is a nucleic acid and the biomarker is a protein, an exonuclease can be employed to release the biomarker.
iv) Photolysis may be used to induce a chemical cleavage in the capture agent, or to cause release of the biomarker from the capture agent. A chemical cleavage site may be the biomarker binding site or may be the surface binding site. Cleavage of a biomarker binding site may serve to release the biomarker from the capture agent. Cleavage of a surface binding site may serve to release the capture agent from the surface. The released capture agent may be unbound or may be bound to a biomarker. Photolysis may comprise exposing the detection area to light of a suitable wavelength to induce cleavage, for example UV light.
v) Addition of competitive binding agents to displace the biomarker from the capture agent and/or the capture agent from the surface.

Therefore, a method of the invention may comprise changing the chemical environment of a detection area; applying light to the detection area wherein the light is of a wavelength suitable to induce cleavage; and/or applying an enzyme to the detection area.

Alternatively, the conditions (environment) of the surface can be modified to provoke a release event.

A rate of change of the surface environment is adjusted to provide a desired rate of release. A change in the environment can be controlled by factors such as the rate of application of reagents such as buffers, salts, acids, or enzymes; altering the concentration of reagents or enzymes, or the power density or intensity of light. It may be preferable to apply any reagent, enzyme or light at a low concentration, intensity or rate to limit the release rate, and optionally increase one or more such factors if an increase in release rate is desired. For example, the rate of diffusion of regents such as enzymes from site to site can be maximised to ensure the desired rate of release is generated. Stochastic release of a release agent may be restricted to a pre-defined detection area. Where release is effected by light, for example photolysis, it may be desirable to couple the photoactivation light to the observation light or optics.

Step IV of any of the methods defined herein involves releasing a particle bound to the surface, wherein the particle is selected from any one or more of:
i. the biomarker released from the capture agent to which it is bound;
ii. a complex comprising the biomarker bound to the capture agent; and/or
iii. unbound capture agent.

Step IV may therefore involve a release of all possible types of particle, and the methods described herein are sufficiently sensitive to distinguish between each these release events. This, it will be possible to determine, for example, how many events release a biomarker bound to a capture agent and how many events release a capture agent alone.

Results/Detection

A baseline measurement may be taken prior to the release event as previously discussed.

Detection of a release event may be performed using a light scattering microscope, for example an iSCAT with a spatial filter or a mass photometer, for example as described herein. iSCAT comprises determining interference between light scattered by an object in a sample and light reflected from the sample location. The interference is dependent on the scattering amplitude of the object, and is measured as an iSCAT signal. It is the monitoring of the mass of this object that permits detection of the release event.

Detection is performed by capturing an image of the surface. Multiple images, or frames, may be combined to provide a film. A video or film may comprise 1,000 to 48,000 frames. It may comprise up to 60,000 frames or up to 100,000 frames. Multiple images are preferred, since this permits each object to be monitored for a release event.

An initial measurement or measurements are taken prior to the application of the release agent. As discussed previously, this can be at the point in the process when the surface has been contacted with the sample and any washing steps have been conducted. This initial measurement provides information on any objects present at the surface. Such objects may be either unbound capture agents or capture agents with bound biomarker.

It is not possible from this initial measurement to directly determine the identity of the objects at the surface. Mass of the objects can be calculated using standard techniques as described herein, including the use of calibration curves.

An initial detection (or further measurement) may be taken immediately upon application of a release agent, or within 5 seconds or less, a second or fraction of a second thereof. However, if the release agent takes a defined time to act, the initial detection can be optimised to take account of this defined time. Alternatively, if the release agent acts immediately, as for example in photolysis, the light for release and detection can be applied in parallel (one wavelength for release, another for detection), with detection taken immediately. Measurements in the form of image capture may be taken at intervals of 0.01 to 1 second. The frame capture rate by the microscope camera may be 0.01 to 1 per second. The frame capture rate may correspond to the detection or measurement rate or time interval.

Detection by image capture may occur effectively continuously for a time period, or may occur at regular or irregular intervals. Such continuous measurement can start prior to application of the release agent and continue for any appropriate period of time. It will be appreciated that the interval between measurements may relate to the particle under investigation, and therefore could be longer. Additionally, the time intervals can be varied within one assay. For example, the first few measurements can be taken every second, and further measurements can be taken with a longer time interval of minutes.

A skilled person can adjust the release rate by adjusting the release agent as discussed herein, and can adjust the frame capture rate, to result in a suitable detection rate.

Each release event involves the departure of a particle from the surface into solution. This is detected by a change in the light scattering of the object at the surface. The change is correlated to the mass of the particle released. The lack of a release event from objects may also be detected, and such may indicate lack of presence of the biomarker in the sample.

Each release event can be assigned a mass, based upon the light scattering. A method of the invention may therefore comprise direct quantitation of bound versus unbound capture agent, and/or biomarker. Thus, the present invention can provide a measurement of the proportion of capture agents that bound a biomarker. A biomarker present in low abundancy in a sample may result in unbound capture agents on the surface. Such can be determined by the methods of the present invention, since discrimination between bound and unbound capture agents is made. A method of the invention may comprise determination of biomarker concentration, by comparison with a known standard.

The method may also be used to analyse heterogenous populations of biomarkers. A method of the invention comprises assigning a mass to each release event. The mass of a biomarker, or the comparison of the mass of abound capture agent versus an unbound capture agent may be used to determine the nature of a biomarker by comparison of the mass to an expected mass.

The method is not suitable only to confirm the presence or absence of a specific biomarker. It may also be used to determine the status of the biomarker, such as the presence of post translational modifications or the presence of the biomarker in complexes. The method is therefore suitable for the relative quantification and determination of the presence/absence of specific protein oligomerization, protein-nucleic acid interactions, protein-sugar or polysaccharide interactions. Thus, many different biological interactions may be scrutinised. Post-translational modifications may be determined simply by determining the mass of the biomarker or by using capture agents specific for differently modified biomarkers.

The method of the present invention is also suitable for the relative quantification of molecules against other internal standards or between two of more biomarkers of interest.

An internal control may permit relative quantification of the biomarker in the sample, for example: an internal standard may be a biomolecule of defined molecular weight that will be added in a known concentration to the sample. By quantifying the release of the internal control and the release of the biomarker, we would have a relative concentration of the biomarker in the sample.

In this aspect, the internal control has a different molecular weight to the biomarker(s) and does not have any cross-reactivity with these biomarkers.

In some aspects, the internal control would require a specific capture agent, and it would be ideal if the capture agent has the same or similar affinity for the internal standard as the capture agent has for the biomarker. However, if these two affinities are different, the affinity difference is known and can be used in order to determine the relative concentration of biomarker. In such an arrangement, the release process for both the internal control and biomarker is the same.

In some aspects, the internal control may be a second biomarker known to be present in the sample to be analysed. This could be a common biomarker where the concentration is already established, such as albumin in serum. The levels of the second biomarker are disease independent and can be used to determine the concentration of the biomarker of interest.

As used herein an internal control may be called an internal standard.

Detection Apparatus

The method may comprise use of a suitable light scattering microscope, for example an interferometric scattering microscope comprising a spatial filter or a mass photometer. Any suitable apparatus may be used, including the arrangement described below.

A suitable microscope or photometer may comprise: a sample holder for holding a surface in a sample location; an illumination source arranged to provide illuminating light; a detector; and an optical system being arranged to direct illuminating light onto the sample location and being arranged to collect output light in reflection, the output light comprising both light scattered from the sample location and illuminating light reflected from the sample location, and direct the output light to the detector.

A microscope may further comprise a spatial filter positioned to filter the output light, the spatial filter being arranged to pass output light but with a reduction in intensity that is greater within a predetermined numerical aperture than at larger numerical apertures. Such a spatial filter advantageously maximises image contrast, as described in PCT/GB2017/052070, and also in Cole et al (ACS Photonics, 2017, 4(2), pp 211-216).

The light used may be: ultraviolet light (which may be defined herein as having wavelengths in the range from 10 nm to 380 nm); visible light (which may be defined herein as having wavelengths in the range from 380 nm to 740 nm); infrared light (which may be defined herein as having wavelengths in the range from 740 nm to 300 μm). The light is preferably visible light. The light may be a mixture of wavelengths. The illuminating light may be coherent light, provided for example by a laser.

A method of the invention may be performed using a suitable microscope which detects light scattering, preferably single particle light scattering. An exemplary design of an iSCAT instrument is described in Cole et al ACS Photonics, 2017, 4 (2), pp 211-216 and Arroyo et al. Nat Protocols 2016, 617-633. Further details about the instrument are provided in WO2018/011591 and GB2552195. A suitable mass photometer is available from Refeyn Limited, Oxford, UK, for example an OneMP mass photometer.

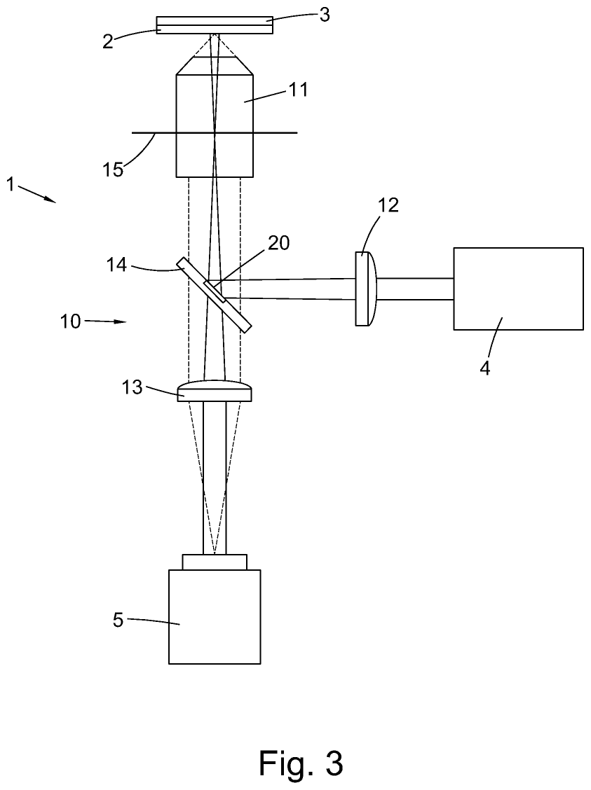

A suitable iSCAT microscope is shown in FIG. 3.

FIG. 3 illustrates an iSCAT microscope 1 which may be employed in the invention which is arranged as follows (and configured with a spatial filter as discussed above). The spatial filter is advantageous for the reasons discussed, to improve contrast, but the method of the invention may alternatively employ an iSCAT microscope without a spatial filter.

The microscope 1 includes the following components that, except for the spatial filter described in more detail below, have a construction that is conventional in the field of microscopy.

The microscope 1 comprises a sample holder 2 for holding a sample 3 at a sample location. The sample 3 may be a liquid sample comprising objects to be imaged, which are described in more detail below. The sample holder 2 may take any form suitable for holding the sample 3. Typically, the sample holder 2 holds the sample 3 on a surface, which forms an interface between the sample holder 2 and the sample 3. For example, the sample holder 2 may be a coverslip and/or may be made from glass. The sample 3 may be provided on the sample holder 2 in a straightforward manner, for example using a micropipette.

The microscope 1 further comprises an illumination source 4 and a detector 5.

The illumination source 4 is arranged to provide illuminating light. The illuminating light may be coherent light. For example, the illumination source 4 may be a laser. The wavelength of the illuminating light may be selected in dependence on the nature of the sample 3 and/or the properties to be examined. In one example, the illuminating light has a wavelength of 405 nm.

Optionally, the illumination light may be modulated spatially, to remove speckle patterns that arise from the coherent nature of the illumination and laser noise, for example as detailed in Kukura et al., “High-speed nanoscopic tracking of the position and orientation of a single virus”, Nature Methods 2009 6:923-935.

The detector 5 receives output light in reflection from the sample location. Typically, the microscope 1 may operate in a wide-field mode, in which case the detector 5 may be an image sensor that captures an image of the sample 3. The microscope 1 may alternatively operate in a confocal mode, in which case the detector 5 may be an image sensor or may be a point-like detector, such as a photo-diode, in which case a scanning arrangement may be used to scan a region of the sample 3 to build up an image. Examples of image sensors that may be employed as the detector 5 include a CMOS (complementary metal-oxide semiconductor) image sensor or a CCD (charge-coupled device).

The microscope 1 further comprises an optical system 10 arranged between the sample holder 2, the illumination source 4 and the detector 5. The optical system 10 is arranged as follows to direct illuminating light onto the sample location for illuminating the sample 3, and to collect output light in reflection from the sample location and to direct the output light to the detector 5.

The optical system 10 includes an objective lens 11 which is a lens system disposed in front of the sample holder 2. The optical system 10 also includes a condenser lens 12 and a tube lens 13.

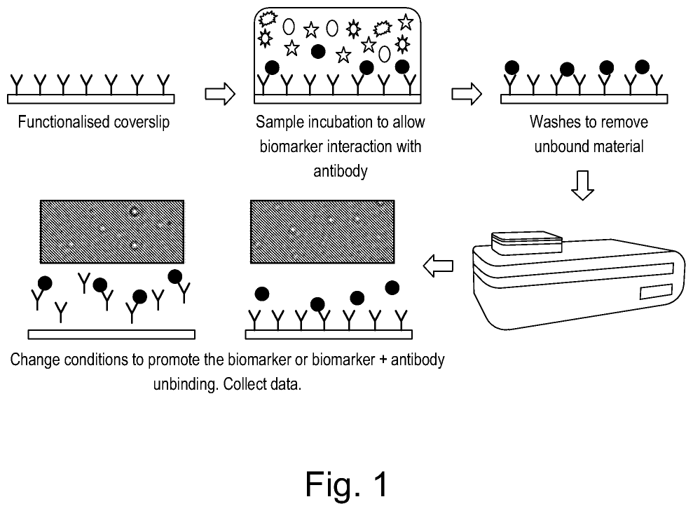

The condenser lens 12 condenses illuminating light from the light source 11 (shown by continuous lines in FIG. 1) through the objective lens 11 onto the sample 3 at the sample location.

The objective lens 11 collects the output light which comprises both (a) illuminating light reflected from the sample location, and (b) light scattered from the sample 3 at the sample location. The reflected light is predominantly reflected from the interface between the sample holder 2 and the sample 3. Typically, this is a relatively weak reflection, for example a glass-water reflection. For example, the intensity of the reflected illuminating light may be of the order of 0.5% of the intensity of the incident illuminating light. The scattered light is scattered by objects in the sample 3.

In a similar manner to conventional iSCAT, scattered light from objects at or close to the surface of the sample constructively interfere with the reflected light and so are visible in the image captured by the detector 5. This effect differs from a microscope operating in transmission wherein the illuminating light that reaches the detector is transmitted through the depth of the sample leading to a much smaller imaging contrast.

The reflected illuminating light and the scattered light have different directionalities. In particular, the reflected illuminating light has a numerical aperture resulting from the geometry of the beam of light output by the light source 4 and the optical system 6. The scattered light is scattered over a large range of angles and so fills larger numerical aperture than the reflected illuminating light.

The tube lens 13 focuses the output light from the objective lens 11 onto the detector 5.

The optical system 6 also includes a beam splitter 14 that is arranged to split the optical paths for the illuminating light from the light source 4 and the output light directed to the detector 5. Except for the provision of a spatial filter as described below, the beam splitter 14 may have a conventional construction that provides partial reflection and partial transmission of light incident thereon. For example, the beam splitter 14 may be a plate, typically provided with a film, which may be metallic or dielectric, arranged at 45° to the optical paths. Alternatively, the beam splitter 14 may be a cube beam splitter formed by a matched pair of prisms having a partially reflective film at the interface between the prisms. Alternatively, the beam splitter 14 may be a polarising beam splitter, used in combination with a quarter wave plate between the beam splitter 14 and the sample 3.

In an example, the light source 4 is offset from the optical path of the objective lens 11 so that the illuminating light from the light source 4 is reflected by the beam splitter 14 into the objective lens 11, and conversely the detector 5 is aligned with the optical path of the objective lens 11 so that the output light from the sample location is transmitted through the beam splitter 14 towards the detector 5.

In addition to the components described above that may be of a conventional construction, the microscope 1 includes a spatial filter 20. The spatial filter 20 is formed on the beam splitter 14 and is thereby positioned behind the back aperture of the objective lens 11, and so directly behind the back focal plane 15 of the objective lens 11. Thus, the spatial filter 20 may be implemented without entering the objective lens as in phase contrast microscopy. Placing the spatial filter directly behind the entrance aperture of the objective rather than in a conjugate plane (for example as described below) has the distinct advantage of strongly suppressing back reflections originating from the numerous lenses within high numerical aperture microscope objectives. This, in turn, reduces imaging noise, lowers non-interferometric background and reduces the experimental complexity, number of optics and optical pathlength leading to increased stability of the optical setup and thus image quality.

However this location is not essential and a spatial filter having an equivalent function may be provided elsewhere as described below.

The spatial filter 20 is thereby positioned to filter the output light passing to the detector 5. In an example in which the detector 5 is aligned with the optical path of the objective lens 11, the spatial filter 20 is therefore transmissive.

The spatial filter 20 is partially transmissive and therefore passes the output light, which includes the reflected illumination light, but with a reduction in intensity. The spatial filter 20 is also aligned with the optical axis and has a predetermined aperture so that it provides a reduction in intensity within a predetermined numerical aperture. Herein, numerical aperture is defined in its normal manner as being a dimensionless quantity characterising a range of angles with respect to the sample location from which the output light originates. Specifically, the numerical aperture NA may be defined by the equation NA=n·sin(θ), where θ is the half angle of collection and n is the refractive index of the material through which the output light passes (for example the material of the components of the optical system 6).

The spatial filter 20 provides no intensity reduction outside the predetermined numerical aperture. In principle, the spatial filter 20 could alternatively provide a reduction in intensity outside its predetermined aperture, but a reduction in intensity that is less than the reduction in intensity within the predetermined numerical aperture, although this is less desirable.

The spatial filter 20 may be formed in any suitable manner, typically comprising a layer of deposited material. The material may be, for example, a metal such as silver. The deposition may be performed using any suitable technique.

As sub-diffraction sized objects near an interface scatter light preferentially into a larger numerical aperture than the reflected illuminating light, the reduction in intensity provided by the spatial filter 20 preferentially reduces the intensity in detection of the reflected illuminating light over the scattered light. Accordingly, the reduction in intensity by the spatial filter 20 at low numerical apertures predominantly affects the reflected illuminating light and has a minimal effect on the scattered light, thereby maximising the contrast in the capture image. The enhanced imaging contrast enables high contrast detection of objects that are weak scatterers.

The contrast enhancement may be understood as follows. As the spatial filter 20 passes part of the output light in the predetermined numerical aperture (i.e. is partially transmissive in this example), fractions of illuminating light and scattered light fields reach the detector and interfere for a sufficiently coherent illumination source. The light intensity reaching the detector I_{det} is then given by I_{det}=|E_{inc}|²{r²t²+|s|²+2rt|s|cosΦ)}, where E_{inc} is the incident light field, r² is the reflectivity of the interface and r² is the transmissivity of the spatial filter 20, s is the scattering amplitude of the object, and Φ is the phase difference between transmitted illuminating light and the scattered light. Thus, the scattering contrast is enhanced, albeit at the expense of the total number of detected photons.

Thus, contrast is provided in a similar manner to conventional iSCAT, but controlled additionally by the transmissivity of the spatial filter. This provides the ability to tune the amplitude of the reference field directly through selection of the transmissivity t² of the spatial filter 20 as opposed to being fixed by the reflectivity of a glass-water interface as in standard iSCAT. In the case that the spatial filter 20 is a layer of deposited material, the transmissivity t² may be selected by choice of the material and/or thickness of the layer. Such tuning may be performed according to, for example, the scattering object of interest, the camera full well capacity and magnification.

To maximise these beneficial effects to iSCAT, the predetermined numerical aperture may be the numerical aperture of the reflected illuminating light within the output light, but that is not essential. For example, benefits of a similar nature could be achieved if the predetermined numerical aperture was slightly smaller than, or larger than the numerical aperture of the reflected illuminating light.

Method of Detection

The detection of biomarker may be carried out using light scattering, for example interferometric scattering microscopy (iSCAT), interferometric scattering mass spectrometry or mass photometry. Suitably, iSCAT or mass photometry is used. iSCAT comprises determining interference between light scattered by an object in a sample and light reflected from the sample location. The interference is dependent on the scattering amplitude of the object (and in turn its polarizability, i.e. volume, density and refractive index)), and is measured as an iSCAT signal. This technique is reviewed for example in Kukura et al., Nature Methods 2009 6:923-935, and in Ortega-Arroyo et al., Physical Chemistry Chemical Physics 2012 14:15625-15636.

Mass photometry is a development of iSCAT (Kukura et al., Nature Methods 2009 6:923-935, and in Ortega-Arroyo et al., Physical Chemistry Chemical Physics 2012 14:15625-15636) and measures the light scattered by single molecules and directly correlates it with molecular mass. The principle of mass photometry is shown in FIG. 4. The light scattered by a particle scales linearly with particle volume and refractive index. As the optical properties and density of proteins vary only by a few percent, their scattering signal is directly proportional to their sequence mass, therefore making it possible to weigh single molecules with light (FIG. X1). The correlation of scattering signal with mass holds true for a variety of biomolecules (glycoproteins, nucleic acids or lipids), making mass photometry a universal analysis tool for biomolecules in solution.

The iSCAT signal may be described as the ratio of detected light in the presence and absence of a particle. In more detail, it may be defined as

(Iₛ−Iₚ)/Iₛ, where Iₛ the reflected intensity from a sample location (such as a glass surface) in the absence of a particle, and Iₚ the same measure in the presence of a particle.

The light scattering signal may be used to assign a mass to the object being detected. Thus, a method of the invention may comprise determining a light scattering signal and using the signal to determine the mass of the particle. The particle is effectively released from the object, and thus a mass can be ascribed to the particle by determining the change in mass of the object. Where mass photometry is used in a method of the invention, the mass is indicated by the mass photometry method.

By comparison with the expected mass of the object to be detected, the presence or absence of the particle can be determined. By measuring the presence/absence of the particle the release event can be detected. Therefore, in detection of a biomarker, the expected mass of a biomarker and/or biomarker/capture agent complex can be compared to the mass of the particle released which is ultimately identified by light scattering, and the presence or absence of a biomarker or biomarker/capture agent complex can be determined. Similarly, the expected mass of the unbound capture agent can be compared to the mass of a particle identified by light scattering and presence or absence of unbound capture agent can be determined. The mass of the biomarker, biomarker/capture agent complex and unbound capture agent may be different. Effectively, the methods of the invention permit the mass of the particle to be measured without specifically interrogating the particle, but by examining the object from which the particle is released.

A method of the invention may further comprise comparison of the iSCAT contrast with a calibration or standard curve to determine the mass or concentration of the particle of interest.

A method of the present invention may also comprise one or more image processing steps, including for example and without limitation removal of the background on the image, and improving the image quality.

The particle is indirectly measured by virtue of its release from an object on the surface. Thus, what is detected and quantified is a “negative mass” event for the object, which can be used to calculate the mass of the particle. The mass of the particle can then determine the presence or absence of the biomarker in that particle.

Once released, the particle or components thereof may bind to the surface. Such binding events may be detected but discounted, since such binding events occur after the release event. This can be seen in particular for Example 1.

Calibration Curve

A calibration curve may be used to determine mass of released particles when iSCAT is used in a method of the invention. A calibration curve may be generated by plotting known mass of two or more standards against scatter values generated by iSCAT. Such a calibration curve may be used to determine the mass of a particle from its scatter value obtained by iSCAT. A method of the invention may include plotting the iSCAT scatter value on a calibration curve to determine the mass of a particle of interest; and optionally charactering the particle, for example as biomarker, capture agent bound to biomarker, or unbound capture agent.

A calibration curve may also be generated to determine the concentration of a particle in a sample.

In one aspect, in order to generate a calibration curve, measurements may be performed with increasing concentrations of a purified biomarker. Such would permit knowledge of the number of unbinding events occurring when incubated with different concentrations of the biomarker. A plot of known unbinding events versus the concentration of biomarker added can permit determination of concentration of the biomarker.

In another aspect, a complex sample (for example serum) can be used and the calibration performed by including a capture agent for a second biomarker that has a known concentration (such as serum albumin). This aspect is described further herein, such that the second biomarker is used as an internal control.

A further calibration method is to perform the method on serial dilutions of the sample, or alternatively using different samples with varying concentration of biomarkers would also allow permit the construction of a plot of concentration versus unbinding.

It will be appreciated by those skilled in the art that the release conditions chosen need to be quantitative over the measurement time. For example, in photolysis the dose can be increased over time ensuring that at the end all cleavable bonds are cleaved. Detection of both bound and unbound capture agents will permit calculation of the ratio of bound: unbound as internal control, and ultimately, biomarker concentration.

Alternatively, an internal control or standard may be used. An internal standard or control may have similar characteristics to a particle such as a biomarker, capture agent or complex of biomarker and capture agent. Internal controls and standards are discussed earlier herein.

Kit

A kit may comprise one or more components suitable for detection of a biomarker by light scattering microscopy. A kit may comprise instructions for use of the kit in accordance with a method of the invention. The instructions may provide reference levels for mass or concentrations of one or more biomarkers, and/or reference single particle histograms for a biomarker. A kit may also comprise details regarding which subjects a diagnostic method may be carried out upon. A kit may comprise one or more items selected from: a suitable surface, e.g. a coverslip, a capture agent, a blocking buffer, a washing buffer, a release buffer, a calibration chart or histogram as described herein, instructions for use in accordance with a method of the invention as described herein, a sample collection container, a sample collection device (such as a capillary blood collection device, a finger prick collection device, or any instrument comprising a needle), an agent to mediate release of the biomarker or capture agent as described herein, and one or more standard biomarker samples for calibration.

The kit may additionally comprise means for the measurement of other laboratory or clinical parameters.

Procedures using these kits may be performed by clinical laboratories, experimental laboratories, medical practitioners, or private individuals.

Additional Methodology

A method of the present invention may comprise one or more further steps.

A diagnostic method according to the present invention may additionally comprise ascribing a diagnosis or prognosis to a subject based upon the results of a method according to the present invention. A diagnostic method may further comprise selecting a suitable treatment regimen or dosage regimen for administration to a subject diagnosed with a disease or condition according to a method of the invention. A method of the invention may comprise administering to a subject a suitable treatment or dosage regimen.

A method of the invention may comprise altering a cell or viral culture method to reduce or increase a particular biomarker of the culture.

A method of the invention may comprise altering a manufacturing process to alter the product, wherein presence, absence or amount of a biomarker for detection by a method of the invention is indicative of a desirable or non-desirable feature of the product, for example quality.

A method of the invention may comprise removing or cleaning a product or culture, for example to remove features or parameters which affect oligomerisation, post translational modification, or biomarker interaction.

Diseases/Infections

A method of the present invention may be suitable for detecting the presence of a biomarker. The biomarker may be indicative of, without limitation, factors such as the presence, type, onset, severity, likely recurrence or recurrence, and progression of a disease or condition, or predisposition to a disease or condition. Diagnosis, as referred to herein, includes testing for presence, type, onset, severity, likely recurrence or recurrence, and progression of a disease or condition, or predisposition to a disease or condition.

Predisposition is the likelihood that the subject will contract the disease or condition, for example a particular time frame. Therefore, the present invention provides a method as described herein for determining predisposition of a subject to a particular disease or condition. The ability of the present invention to detect low abundance biomarkers in biological samples makes it particularly suitable for determining the likelihood that a subject will contract the disease or condition which the biomarker is indicative of. In other embodiments the method is a method for diagnosing a disease in the subject and the presence of the detectable marker in the subject is indicative of the subject having the disease and the absence of the detectable marker in the subject is indicative of the subject not having the disease.

Suitably, a method of diagnosis as described herein is performed on a sample obtained from a subject. A sample may be obtained using a capillary blood collection device, a finger prick collection device, or any instrument comprising a needle. A sample may be collected using any suitable biopsy technique, or collection method including bronchoalveolar lavage, sputum aspiration, swabs and the like and can be placed into any suitable receptacle.

The present invention may be suitable for the diagnosis of a disease or condition which includes, but is not limited to, cancer, a degenerative disease, liver injury or disease, a bacterial, fungal or viral infection, or an inflammatory disease.

The present invention may be suitable for diagnosis of a disease or condition involving any protein modifications, including but not exclusive to diseases caused by: changes in protein oligomerization, protein-protein interaction, protein interactions with other biomolecules (e.g. nucleic acids, sugars, polymers, smaller peptides, organic and inorganic molecules), protein post-translation modification (e.g. changes in the protein phosphorylation, glycosylation, ubiquitination, nucleic acids (e.g. methylation) or polysaccharides (e.g. presence of specific acetyl- or acyl-groups). The present invention may also be suitable for detection of specific bacterial, fungal or viral infections due to the presence/absence of specific biomarkers, including the presence of: specific DNA or RNA sequences; envelop and/or membrane proteins; lipopolysaccharide; effector proteins. The present invention may be suitable for the diagnosis of a cancer by the detection of a carbohydrate, nucleic acid or protein biomarker, or any combination thereof.

Diseases that are associated with protein modifications include prion diseases such as scrapie, scrapie and bovine spongiform encephalopathy of animals and Creutzfeldt-Jakob and Gerstmann-Straussler-Scheinker diseases of humans. Protein aggregates that result in inclusion formation are a pathological hallmark common to many neurodegenerative diseases, including amyotrophic lateral sclerosis, Parkinson's disease and Huntington's disease.

The term “cancer” as used herein refers to proliferative diseases, such as lymphomas, lymphocytic leukaemia's, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumours, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

Inflammatory disease includes, without limitation, conditions such as Cardiovascular Disease, inflammatory Bowel Disease, infection, multiple sclerosis, atherosclerosis, allergic disease, asthma, and CORD.

Autoimmune diseases are a group of diverse diseases with a common etiology in which the immune system responds to self-antigens leading to damage or dysfunction of tissues. Autoimmune diseases including Crohn's disease, ulcerative colitis, rheumatoid arthritis, psoriasis and systemic lupus erythematosus (Norouzinia, et al Gastroenterol. Hepatol. from Bed to Bench 10, 155-167 (2017); Jin, F. et al. Front. Immunol. 9, 1-9 (2018); Shi, G., Zhang, Z. & Li, Q. J. Immunol. Res. 2017, 1-2 (2017); Prince, H. E. Biomarkers 10 Suppl 1, 44-49 (2005)).

Liver damage may include alcohol related liver disease, non-alcoholic fatty liver disease, hepatitis, heamochromatosis and cirrhosis.

A bacterial, fungal, parasitic or viral infection includes for example and without limitation bacterial infections such as Bordetella, Chlamydia, or Mycoplasma, legionella, bacterial meningitis, pneumonia, bronchitis, sepsis, and viral infections such as Coronavirus, Human Immunodeficiency Virus (HIV), Hepatitis B virus (HBV), or Hepatitis C virus (HCV)), HSV, CMV, Rhinovirus, Influenza A, Influenza B, Parainfluenza, or RSV. Fungal infections include, without limitation, Aspergillus, Candida, Penicillin, Paracoccidodiodes, Histoplasma, Fonsecaea, Cryptococcus, Saccaromyces, Pichia, C albicans, C glabrata, C tropicalis, Fusarium spp, Saccaromyces cerevisiae, and Acremonium spp. Parasitic infections include, without limitation, Malaria, Toxoplasmosis, Leishmaniasis, and African Trypanosomiasis. Infection with amoeba may also be detected, for example Naegleria fowleri or Entamoeba histolytica.

### INDUSTRIAL APPLICATIONS

This approach described within the document is not limited to diagnostics applications, but can be extended to any other relevant applications, such as quality control, environmental control and bio-analytical methods. The present invention may have utility in any application which requires the detection and/or quantitation of a biological molecule, such as in the mass production of food and beverages.

Therefore, a method of the present invention may be useful for detecting contamination in a sample or a cell or virus culture, determining the quality of a sample compared to a known standard, detecting modification of a biological molecule (for example post-translational modification of a protein), detecting or quantitating interactions between two or more biological molecules in a sample (for example detecting or quantitating binding or association), comparing the amounts of two or more biomarkers in a sample, quantitating the expression level of a product.

The present invention may also have utility for mass testing procedures such as testing sewage or effluent for outbreaks of infection such as viral infection.

A kit as described herein may be provided for use in such a method.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### EXAMPLES

### Example 1

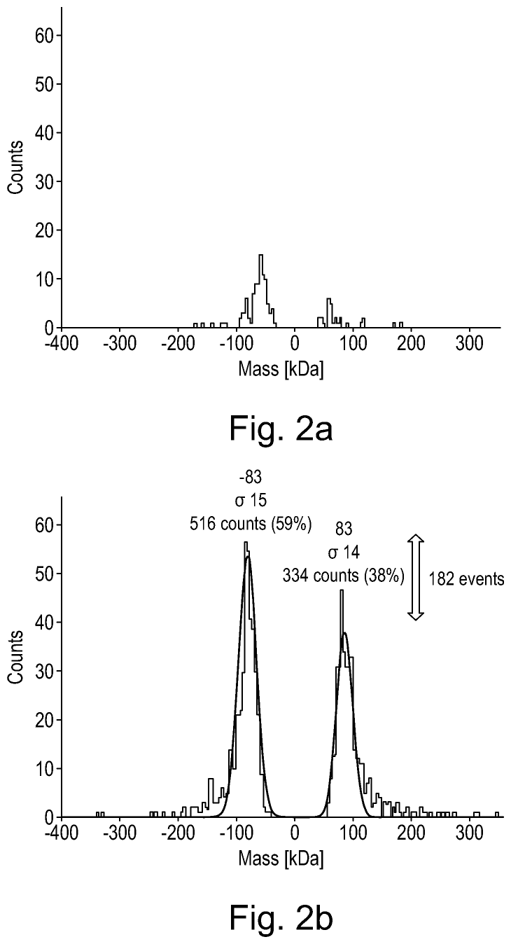

Clean coverslips were incubated with 10 μl of 400 μM solution of antibody (Herceptin) (Hoffmann-LaRoche) for 10 min to allow binding to the coverslip via non-specific absorption. After incubation the excess and unbound antibody was removed by washing the coverslip with TBS buffer. The excess of buffer was removed using a nitrogen air in-line. Then 10 μl of a Her2 (≈500 nM) was added to the coverslip and incubated at room temperature for 20 min to allow the protein to bind to the antibody in the coverslip. After incubation the excess and unbound protein was removed by washing with abundant TBS buffer. The coverslip was dried using nitrogen air in-line and used in mass photometry. AcquireMP (Refeyn, UK) was set to collect 18000 frames of ROI (region of interest) of 512×260 pixels per movie. For the measurements, 10 μl of TBS (pH7.4) was added to the coverslip to find the focus and acquire control data to evaluate random binding/unbinding events due to the coating itself (control experiment, FIG. 2a). Then 5 μl of buffer was removed and 5 μl of hydrochloric acid (1M) added without mixing and a second movie was acquired (FIG. 2b).

Coated coverslips incubated with TBS pH 7.4 show no significant release of proteins from its surface (FIG. 2a).

Addition of HCl to the TBS buffer to decrease the buffer pH induces the release of Her2 protein from Herceptin that mostly remains bound to the surface. It is known that lowering the buffer pH induces antibodies to release bound antigens, which explains Her2 unbinding. The lack of surface passivation allows Her2 to re-bind to available spaces on the glass, which accounts or the high number of binding events in FIG. 2b (334 binding events). Still the number of unbinding events is higher than the binding (516 unbinding events versus 334 binding).

The results show that it is possible to release a breast cancer biomarker (Her2) from an antibody (Herceptin) using conditions that induce that release, furthermore, the results show that mass photometry can be used to evaluate the biomarker unbinding from the antibody and its release can be confirmed by assessing the molecular weight of the Herceptin bound to the surface with or without Her2 before and after the release event. Such is depicted in FIG. 1. The functionalised coverslip with the Herceptin is prepared. This is incubated with the sample which can include many components. Herceptin binds specifically to Her2 (if present) and the other components are washed away. The surface is then introduced to the apparatus, and an image of the surface is taken, which depicts the objects (herein Herceptin with or without the Her2 bound). The conditions are then changed, such that the Herceptin is released from the surface. The change in the light scattering for each object is then determined, and the mass of the particle released can be assigned. The mass will indicate whether it is the Herceptin alone released (no Her2) or Herceptin bound to Her2. The proportion of bound to unbound Herceptin can be used to indicate the concentration of Her2 in the sample.

### Example 2

Summary of the Experimental Set-Up (FIG. 4)

Glass coverslips were coated with a mix of PEG and PEG-biotin (Step 1). PEG is a known anti-fouling coating that minimizes unspecific protein binding to surfaces. The biotin molecule present in PEG-biotin polymer will bind streptavidin upon incubation with this protein (Step 2).

The streptavidin now bound to the coating through the biotin, is used as a capture agent for biotin-labelled proteins, in this case a Herceptin antibody labelled with biotin (Step 3). The presence of PEG in the glass surface minimizes the attachment of other proteins. Accordingly, even if Herceptin-biotin (Herc-B) is incubated in the surface with a complex mix of other proteins, such as serum proteins, the only protein remaining in the glass after incubation and washes would be the labelled antibody, Herc-B (Step 3).

Streptavidin has significant more affinity to free biotin than to labelled molecules, such as the labelled antibodies, therefore, the addition of high concentrations of free biotin to the glass coverslip will promote the release of the antibody from the glass surface (Step 4). This release can be followed by mass photometry by measuring the light scattering at the surface and noting the change of the magnitude of the signal.

Detailed Protocol—Preparation of the Coverslips:

Clean coverslips were coated with PEG and PEG-biotin in the ratio (9:1). The coated coverslips were incubated with streptavidin (50 μl at 200 nM) for 30 min to allow streptavidin binding to the biotin on the coating. After incubation, the coverslips were washed thoroughly and dried.

To determine if the coating would promote selective binding of Herc-B and effectively reduce other proteins binding to the surface, Herc-B was mixed with diluted fetal bovine serum (FBS) before the incubation onto the coverslip. FBS was diluted 2-folds in PBS and mixed with Herc-B to a final concentration of 100 nM of the labelled antibody. The resulting complex and high concentration protein mix was then incubated on the coverslips for 30 min, before being washed to remove unbound protein, dried and used in mass photometry measurements.

Mass Photometry Measurements and Results:

Following the release of Herc-B from the coated coverslips when the Herc-B was part of a complex sample:

The coverslips prepared were introduced into the mass photometer, initial measurements taken and protein released from or binding to the coverslips was followed for 500 seconds using the large FOV.

Briefly, the Measurements were Performed as Follows:

The well was incubated with 20 ul PBS for about 5 min to re-hydrate the surface. After this short incubation, the PBS was removed, fresh PBS was added and a control measurement was performed. Events detected during the base-line or control measurement account for the unspecific unbinding/binding on the coating and background noise. After the control measurement, the PBS was removed, measurements were performed on the same well as free biotin was added to the glass at a concentration of 25 mM.

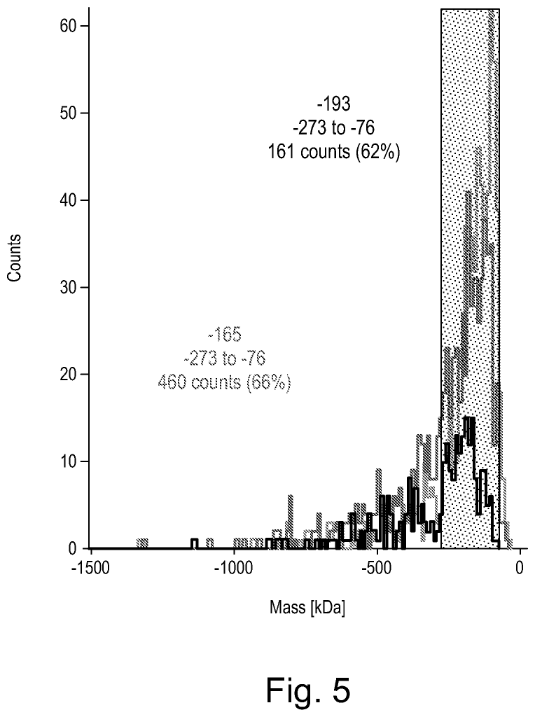

The results (table 1 and FIG. 5) indicate that the presence of biotin significantly increases the overall number of events detected and particularly the number of unbinding events with can be associated with a molecular weight close to the 150 kDa expected for Herc-B antibody (FIG. 5). Table 1 summarises the number of binding and unbinding events detected within the range of 76 to 273 kDa, assuming that the release of various particles would be detected within this range.

The data shows that the first 500 seconds after the free biotin addition promoted the release of 460 events within Herc-B MW range (dark grey line on FIG. 5). The consecutive measurement presented more counts, but the difference between the number of binding (not shown) and unbinding events (FIG. 5, light grey line) was smaller, indicating that most of the unbinding events happened within the first 500 seconds. Both time points have a higher difference of binding to unbinding events than the PBS control, indicating that the biotin promotes a controlled release of the labelled antibody (table 1).

Furthermore, most of the release events detected using biotin enables a calculation that the particles released have a molecular weight close to the expected molecular weight release expected for Herc-B release, indicating that the coating has effectively captured the antibody from a complex, high concentration protein mix, composed by common protein present in the bovine serum (very high concentrations) and Herc-B, mixed in a low concentration (100 nM).

FIG. 5 depicts only the negative mass events detected, i.e. the unbinding events. The negative mass is detected since the complex (object) at the surface is monitored for release of the particle (in this case Herc-B). Given that the molecular weight of this antibody is known, it is possible to monitor for unbinding events that involve a release of the expected mass from the surface. FIG. 5 is a histogram of mass released from the surface (kDa) versus counts. Three sets of data are depicted—the dark grey and light grey lines lie indicates the results achieved with 25 mM Biotin (2 repeats). The darkest line indicates the results with PBS used instead of biotin.

TABLE 1
Summary of the number of events binding and unbinding from the
glass which can be correlated to a molecular weight between 76
and 273 kDa, the difference between the number of unbinding and
binding events and the increase of the unbinding the binding events
compared to the control (PBS) calculated as a percentage.

| | | Events which can be | | | |
|---|---|---|---|---|---|
| | | assigned to particle | | | |
| | Shade of | with a MW between 76 | | | |
| | line on | to 273 kDa (counts) | | | Percentage |
| Release | histogram | Binding | Unbinding | Difference | increase |
| Agent | (FIG. 5) | (B) | (UB) | UB − B | to control |
| PBS | Black | 144 | 161 | 17 | — |
| (control) | | | | | |
| Biotin 1 | Dark grey | 281 | 460 | 179 | 90.5% |
| Biotin 2 | Light grey | 481 | 565 | 84 | 79.8% |

This Example, therefore, demonstrates the ability of biotin to be used as a release agent in the methods of the invention, and also the utility of biotinylated PEG to immobilise the streptavidin capture agent. Although binding events were recorded, this is not critical for the methods of the invention. The release of the antibody can be detected as a release event and the magnitude of the change at the surface can be used to allocate a molecular weight to the particle.

1. A method for detecting a biomarker in a sample by interferometric light scattering or mass photometry, wherein the method comprises:
I) providing in solution a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface and taking a measurement of the surface using light scattering;
IV) releasing a particle bound to the surface, wherein the particle is selected from:
i. the biomarker released from the capture agent to which it is bound;
ii. a complex comprising the biomarker bound to the capture agent; and/or
iii. unbound capture agent;
V) detecting the particle(s) released from the first detection area of the surface by determining the change in light scattering at the surface,

2. A method according to claim 1 wherein step V) comprises determining the loss of mass from the surface.

3. A method according to claim 2 where the loss of mass from the surface allows determination of the identity of the particle.

4. A method according to any one of claims 1 to 3 wherein step IV) comprises application of a release agent to a detection area of the surface.

5. A method according to claim 4 wherein step IV) comprises altering the chemical environment of a detection area of the surface; enzymatic digestion of the capture agent, and/or photolysis of the capture agent.

6. A method according to claim 4 or 5 wherein the release agent is an enzyme, light, a buffer or a chemical reagent.

7. A method according to any one of claims 1 to 6, wherein a detection area of the surface is passivated, activated, coated, treated or derivatised.

8. A method according to any one of claims 1 to 7 for measuring the concentration of a biomarker, wherein the method comprises i) determining the expected mass of a particle of claim 1, ii) determining the number of particles of the expected mass or contrast released from the surface; and optionally iii) comparing the result of ii) to a standard or calibration curve.

9. A method according to any one of claims 1 to 7, comprising repeating steps IV) and V) for a second or further detection area of the surface.

10. A method of diagnosing a disease or condition associated with presence or amount of a biomarker in a subject by interferometric light scattering or mass photometry, wherein the method comprises:
I) providing in solution a surface comprising a capture agent immobilised thereon wherein the capture agent is capable of binding to the biomarker present in the sample;
II) contacting the surface with the sample under conditions to allow binding of biomarker in the sample to the capture agent;
III) defining a first detection area of the surface and taking a measurement of the surface using light scattering;
IV) releasing a particle bound to the surface, wherein the particle is selected from:
i. the biomarker released from the capture agent to which it is bound;
ii. a complex comprising the biomarker bound to the capture agent; and/or
iii. unbound capture agent;
V) detecting the particle(s) released from the first detection area of the surface by determining the change in light scattering,
wherein the presence or absence of i) or ii) is indicative of the presence, severity, or likelihood of developing the disease or condition in the subject.

11. A method according to claim 10, wherein method as defined in any one of claims 1 to 9.

12. A method according to any one of the previous claims, further comprising:
i) providing a surface;
ii) providing a sample to be analysed for presence and/or amount of a biomarker;
iii) immobilising on the surface a capture agent which specifically binds to the biomarker to be detected.

13. A method according to any one of the previous claims wherein step II) comprises incubating the surface with the sample for a suitable time period and under suitable conditions to allow binding of a biomarker present in the sample to the capture agent immobilised on the surface.

14. A method of selecting a subject to whom a substance or composition is to be administered, or to whom a treatment or dosage regimen is to be prescribed, wherein said substance or composition or regimen is suitable for treating or preventing a disease or condition associated with the presence or amount of a biomarker in a sample from the subject, the method comprising a method of diagnosis as defined in any one of claims 10 to 13.

15. A method of treating or preventing a disease or condition in a subject diagnosed with a disease or condition or likelihood of developing said disease or condition in the subject according to any one of claims 10 to 13 wherein the method comprises administering a substance or composition to the subject, or carrying out a regimen thereon, which is effective to treat or prevent said disease or condition in the subject.

16. A substance or composition for use in a method of treating or preventing a disease or condition in a subject diagnosed with a disease or condition or likelihood of developing said disease or condition in the subject according to any one of claims 10 to 13.

17. A method according to any one of the previous claims wherein the biomarker is a protein and a fragment thereof, a peptide, a polypeptide, a proteoglycan, a glycoprotein, a lipoprotein, a carbohydrate, a lipid, a nucleic acid, an organic on inorganic chemical, a natural polymer, or a small molecule, or a combination thereof.

18. A method according to any one of the previous claims, wherein the capture agent is an antibody or a derivative thereof, a nucleic acid, a protein, an inorganic molecule or a polymer.

19. A method according to any one of the previous claims wherein the capture agent comprises one or more cleavage site, for chemical digestion, digestion by an enzyme or cleavage by photolysis, to release a biomarker from the capture agent and/or a capture agent from the surface.

20. A method of detecting modification of a biological molecule, comprising a method according to any one of claims 1 to 9, wherein the biomarker is indicative of the presence of the modified biological molecule.

21. A method for detecting interactions between two or more biological molecules in a sample comprising a method according to any one of claims 1 to 9, wherein the biomarker is indicative of the presence of an interaction between the two or more biological molecules.

22. A method of detecting contamination in a sample, comprising a method according to any one of claims 1 to 9, wherein the biomarker is indicative of contamination of the sample.

23. A method of quantitating the expression level of a product, comprising a method according to claim 8, wherein the biomarker is indicative of is indicative of presence of the expression product.

24. A kit comprising a surface, a capture agent, a buffer, instructions for use in accordance with a method as defined in any one of claims 1 to 23, a sample collection device, one or more standard biomarker samples for calibration, and optionally an agent to mediate release of the biomarker or capture agent from a surface.

25. A kit according to claim 24 wherein the agent is a chemical reagent or an enzyme.
